(19) United States
(12) Patent Application Publication
(10) Pub. No.: US 2011/0287093 A1
(43) Pub. Date: Nov. 24, 2011

(54) ORAL DOSAGE FORMS WITH THERAPEUTICALLY ACTIVE AGENTS IN CONTROLLED RELEASE CORES AND IMMEDIATE RELEASE GELATIN CAPSULE COATS

(76) Inventor: Grant L. Schoenhard, San Carlos, CA (US)

(21) Appl. No.: 12/754,486

(22) Filed: Apr. 5, 2010

Related U.S. Application Data
(63) Continuation of application No. 11/831,741, filed on Jul. 31, 2007, now abandoned.
Application No. 11/831,741 is a continuation of application No. 10/742,672, filed on Dec. 18, 2003, now abandoned.
(60) Provisional application No. 60/434,839, filed on Dec. 18, 2002.

(51) Int. Cl. A61K 9/52; A61P 29/00

(52) U.S. Cl. 424/456

(57) ABSTRACT

The present invention relates to oral dosage form with active agents in controlled release cores and in immediate release gelatin capsule coats.

### CROSS REFERENCED APPLICATIONS

None applicable.

### BACKGROUND OF THE INVENTION

The maximum time of effectiveness of many oral dosage forms is only a few hours. While it is often desirable to reach an effective dose quickly, in order to maximize patient compliance, it is also considered desirable to reduce the frequency of dosing, thereby reducing the number of doses a patient must take in order to attain effective therapy. In the case of combination therapy where two drugs may be given in the same dosage form (e.g., tablets, capsules, etc.), the frequency of dosing is further reduced.

For any given dosage form of an agent, such as a drug, the amount of the agent from the dosage form that is available to reach the circulation system depends first on the rate and extent of release from the dosage form. Following oral administration, drug or prodrug is released from the dosage form containing the drug or prodrug in the gastrointestinal (GI) tract and free drug is absorbed. The extent of release determines the amount of drug available for absorption. The rate of release gives the amount of drug available for absorption per unit time. Drug dosage forms that rapidly release the drug into the GI tract are termed immediate release or IR formulations. The time, tₘₐₓ to reach to maximum plasma concentrations (Cₘₐₓ) of the drug in the body ranges from a few minutes to two plus hours for such immediate release formulations. During the absorption phase, the drug is distributing throughout the body, and in most cases are beginning and/or simultaneously being eliminated from the body. Thus, the pharmacokinetic profile (the graph of drug in blood or plasma concentration vs. time) for repeated administration of immediate release formulations cycle from minimum or trough plasma concentrations Cₘᵢₙ to peak plasma concentrations, Cₘₐₓ, and back to minimum or trough plasma concentrations, Cₘᵢₙ.

To achieve sustained concentration of circulating drug or active metabolite(s) or conjugate(s) of drug over a longer period of time between doses, controlled release (alternative constant release (SR) or extended release) drug formulations were developed. These controlled release (CR) formulations require approximately from 2 to 3 hours to achieve Cₘₐₓ and the minimum effective concentration (MEC) of drug in the circulation, and can maintain MEC levels from about 12 to about 22 hours before declining exponentially because no more drug is being released from the dosage form and systematically absorbed. Thus, the pharmacokinetic profile of controlled release formulations have a shape similar to a hyperbole, with a slow and gradual increase in drug blood levels to a plateau, followed by a decline in plasma concentrations.

When comparing the pharmacokinetic profiles of immediate release with controlled release drug formulations, there are two major differences. First, the time to achieve the Cₘₐₓ in the plasma is often longer in the controlled release versus the immediate release formulation. In controlled released formulations, a long tₘₐₓ is particularly disadvantageous to patients seeking urgent treatment and to maintain MEC levels. A second difference in the pharmacokinetic profiles of controlled release in comparison to immediate release drug formulations is that the duration of sustained plasma levels is longer in the controlled release formulations. The longer duration of such sustained plasma levels facilitated by controlled release formulations are advantageous to all patients, prolonging the desired biological effect. Therefore, although the controlled release formulation facilitates a substantially longer period of time in maintaining plasma levels of drug or active metabolite(s), it suffers from the drawback of requiring longer periods of time to achieve the Cₘₐₓ, when compared to immediate release formulations. Thus, there remains a long felt need for improved controlled release formulations, including dosage formulations that might have one or more desirable characteristics of both immediate release and controlled release formulations.

### SUMMARY OF THE INVENTION

The present invention is directed to novel oral dosage forms with therapeutically active agents in both controlled release cores and immediate release gelatin capsule coats. The agents have different release profiles from the cores and gelatin capsule coats. The controlled release cores optionally comprise additional components for the immediate release of a portion of therapeutically active agent from the core. Such gelatin capsule encapsulated controlled release oral dosage forms constitute improved controlled release dosage forms and achieve a rate of release and an extent of release not previously achieved by either immediate release or controlled release dosage forms of therapeutically active agent(s). Soft gelatin capsules, such as softgels, with at least one therapeutically active agent are preferred for encapsulating the cores. The invention further relates to pharmaceutical formulations useful in the preparation of such dosage forms, as well as to methods of making and administering such dosage forms. Gelatin capsule encapsulated controlled release cores of at least one therapeutically active agent, including liquid, tablet, or solid cores, wherein the gelatin capsule encapsulating such controlled release core comprises an immediate release formulation of at least one therapeutically active agent are improved dosage forms with surprising advantages. Such gelatin capsule encapsulating wherein the gelatin capsule contains at least one therapeutically active agent, enables the increase of the rate of release of the therapeutically active agent(s) from oral dosage forms of the invention and/or increases the apparent extent of exposure to sustained blood/plasma concentrations of the agent(s) and/or metabolites or conjugates of such agent(s), as well as the related pharmacodynamic response, for example, when at least one of the therapeutically active agents is the same in the gelatin capsule coating and in the core.

Novel oral dosage forms according to the invention comprise (i) an controlled release core, and (ii) an immediate release gelatin capsule around the controlled release core, wherein the controlled released core comprises at least one therapeutically active agent and at least one controlled release material, and wherein the immediate release gelatin capsule comprises at least one therapeutically active agent. Such oral dosage forms have at least one therapeutically active agent in the controlled release core that is the same as or different from at least one therapeutically active agent in the immediate release gelatin capsule. Preferred dosage forms according to the invention have the same therapeutically active agent in both the core and the immediate release gelatin capsule and optionally may have other agents in either or both of the core and gelatin capsule. Such gelatin capsule encapsulated controlled release dosage forms as described herein, achieve an increased rate of release of the therapeutically active agent via the immediate release gelatin capsule and an increased extent of duration of exposure to stable blood/plasma concentration of the therapeutically active agent(s) and/or active metabolite(s) or conjugate(s) via the combination of the release of active agents from the immediate release gelatin capsule and the controlled release core, with initial and repeated administration of the dosage form. Following repeated administration to a subject, dosage forms according to the invention provide immediate and continual release of active agent(s), such as a drug, for absorption by distribution of and elimination from the subject, and can maintain the desired pharmacokinetic and/or pharmacodynamic profiles. Optionally, the controlled release core with at least one therapeutically active agent and at least one controlled release material, can further comprise immediate release components having at least one therapeutically active agent, wherein, for example, the components are in the form of a liquid, granulate, particulate, pellet, or bead. Preferably, at least one agent in the immediate release components of the core is the same as at least one agent in the controlled release components of the core and/or in the immediate release gelatin capsule coat.

The present invention provides novel oral dosage forms with unexpectedly superior results using a liquid (including, for example, a high viscosity liquid) or solid (including, for example, a granulate, particulate, pellet or bead) controlled release formulation with at least one therapeutically active agent and at least one controlled release material as a controlled release core. Thus, the core may be, for example, a liquid, tablet or capsule. This core is coated by encapsulating such core with an gelatin capsule, preferably a soft gelatin capsule, that also contains at least one therapeutically active agent as an immediate release formulation. Preferably, at least one therapeutically active agent that is in the core as a controlled release formulation is the same agent that is in the immediate release gelatin capsule coating as an immediate release formulation. A multiplicity of controlled release materials are known and useful according to the invention, including, for example, high viscosity liquid carrier materials (HVLCM) as described herein and in U.S. Pat. Nos. 5,747,058; 5,968,542; 6,413,536; and corresponding PCT publications WO 96/39995; WO 99/13913; WO 01/15734, such as, for example, sucrose acetate isobutyrate (SAIB).

Therapeutically active agents suitable for dosage forms of the invention include biologically active substances that are useful for human therapy, veterinary therapy, or for agricultural purposes. Therapeutically active agents include organic molecules, for example, drugs. Drugs include substances used as medicines for the treatment (e.g., prophylactic or therapeutic), cure or prevention of a disease, condition or disorder. Drugs include prodrugs. Among the preferred therapeutically active agents suitable for dosage forms according to the invention are analgesics, including opioids. Among the particularly preferred therapeutically active agents suitable for such dosage forms are opioid agonists, alone or in combination with opioid antagonists. The present invention thus provides controlled release pharmaceutical formulations in the form of a liquid, tablet, or capsule as a controlled release core, wherein the core comprises one or more therapeutically active agents and one or more controlled release materials. Optionally, the core additionally comprises one or more therapeutically active agents and one or more immediate release components. Preferably, at least one active agent is in both a controlled release and an immediate release form in such a core. A core is then encapsulated with an immediate release gelatin capsule comprising immediate release pharmaceutical formulations of one or more therapeutically active agents. The effect of such novel dosage forms is to increase the rate of release of the therapeutically active agent(s) from the dosage form via the immediate release gelatin capsule coating comprising the therapeutically active agent(s), and to increase the apparent extent of exposure to sustained blood/plasma concentration(s) of the therapeutically active agent(s) or active metabolic or conjugate thereof, from the dosage form (via both the immediate release gelatin capsule coating and the controlled release core each comprising therapeutically active agent(s). The combination of gelatin capsule coating and controlled release core (with or without additional immediate release components in the core) achieves the increased rate and extent/duration of release with initial and repeated administration of the dosage form as well as the related pharmacodynamic response. Such dosage forms according to the invention are administrable at least every 8 hours and preferably administrable once-a-day (every 24 hours) or twice daily (every 12 hours). A preferred dosage form according to the invention comprises (i) a controlled release core that has an opioid agonist, such as, for example, oxycodone or morphine, as a therapeutically active agent, alone or in combination with an opioid antagonist, such as, for example, naltrexone or nalmefene, and a controlled release material, such as, for example, SAIB, and (ii) an immediate release gelatin capsule, including a soft gelatin capsule, for example, such as softgels, enteric softgels, or gelcaps, that has an opioid agonist, for example, such as oxycodone or morphine, as a therapeutically active agent, alone or in combination with an opioid antagonist, for example, such as naltrexone or nalmefene. Preferred manufacturers of gelatin capsules containing no active agent in the gelatin capsule are Banner Pharmacaps (see, e.g., their Softgel, Gelatin Binary System™, and Sofia™ Gelcap products) and Cardinal (see, e.g., their LIQUI-GELS®, RP SCHERERSOL®, and PULSIN-CAP® technology and products). Novel gelatin capsules may be prepared according to the invention by incorporating at least one therapeutically active agent in a gelatin formulation that is used to encapsulate an core according to the invention.

### DESCRIPTION OF THE FIGURES

The detailed description of the invention will be made with reference to the accompanying drawing, where like numerals designate corresponding parts of the figures. The drawings are meant to be generally illustrative of various examples of the present invention, but are merely example and are not meant to be limiting the scope of the invention.

FIG. 1 is a release profile of a traditional controlled release formulation or dosage form of therapeutically active agent.

FIG. 2 is a release profile of a formulation or dosage form according to the present invention, illustrating an increased rate of release and an increased apparent extent of exposure to sustained blood/plasma concentrations of therapeutically active agent as compared with a traditional controlled formulation/dosage form.

FIG. 3 is the chemical structure of SAIB, sucrose acetate isobutyrate.

### DETAILED DESCRIPTION OF THE INVENTION

The present invention generally relates to an oral dosage form comprising (i) a controlled release core; and (ii) an immediate release gelatin capsule that encapsulates controlled release core, wherein the controlled released core comprises at least one therapeutically active agent and at least one controlled release material, and wherein the immediate release gelatin capsule comprises at least one therapeutically active agent. Such novel oral dosage forms represent improved controlled release dosage forms. The dosage forms and formulations presented herein achieve an increased rate of release of the therapeutically active agent and an increased apparent extent of exposure to sustained blood/plasma concentrations of the therapeutically active agent and/or its active metabolite(s) and/or conjugates via the combination of the immediate release gelatin capsule and the controlled release core with initial and repeated administration of the dosage form. Optionally, the controlled release core can also contain an immediate release components in the form of, for example, liquids, granulates, particulates, pellets, beads, etc.) also comprising a therapeutically active agent. Preferably, at least one therapeutically active agent is the same as in the controlled release core and/or the immediate release gelatin capsule.

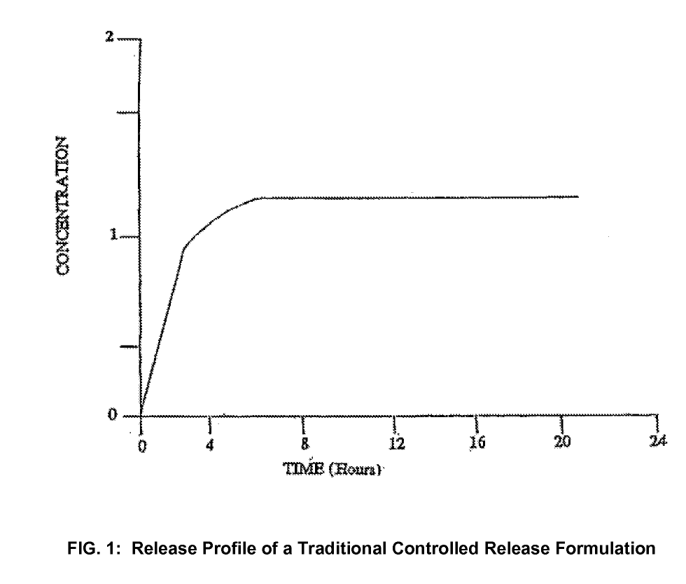

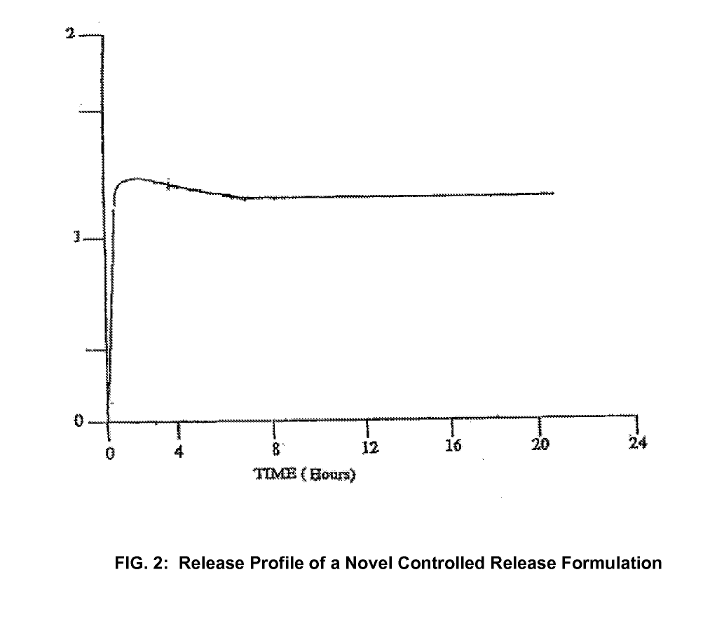

The release profile of traditional controlled release formulations or dosage forms, as shown in FIG. 1, generally have a shape similar to a hyperbole, with a slow and gradual increase in blood/plasma levels of an active agent such as a drug, to a plateau, followed by a decline in blood/plasma concentrations. In contrast, FIG. 2 show the release profile of a formulation or dosage form according to the present invention with active agent in both controlled release core and immediate release gelatin capsule illustrating a rapid and increased rate of release of active agent, as well as an increased apparent extent of exposure to sustained blood/plasma concentrations of the active agent or active metabolite(s) thereof from initial and repeated administration of the dosage form. Such increased rate and extent of release and exposure results in an increase in the related pharmacodynamic response.

The invention provides surprisingly and unexpectedly superior results using a liquid semi-solid or solid (including, without limitation, particulates, granules, or beads) controlled release formulation with at least one therapeutically active agent as an core that is coated with an gelatin capsule by encapsulating wherein the gelatin capsule also contains at least one therapeutically active agent as an immediate release formulation. Preferably, at least one therapeutically active agent present in the core as a controlled release formulation is the same as at least one therapeutically active agent present in the immediate release gelatin capsule coating as an immediate release formulation. Optionally, the core can additionally contain a portion of immediate release components, in the form of, for example, liquids, granulates, pellets, or beads, each comprising at least one therapeutically active agent. Again, preferably the immediate release component of the controlled release core comprises at least one therapeutic agent that is the same as the agent in the controlled release portion of the core and/or the same as the agent in one gelatin component.

The invention provides liquids or liquid gels of varying viscosity, as well as tablets or capsules that comprise an controlled release core with at least one controlled release material and at least one therapeutically active agent, wherein the liquid, tablet, or capsule core is coated by an gelatin capsule. The gelatin capsule encapsulates the core. Encapsulating includes coating, covering, encasing, enrobing, enveloping and capsuleing. This immediate release gelatin capsule comprises an immediate release formulation of at least one therapeutically active agent, preferably the same therapeutically active agent that is in the controlled release core. The invention thus provides an controlled release core comprising a therapeutically active agent in the form of a liquid, tablet, or capsule that is encapsulated with an immediate release gelatin capsule coating of the same therapeutically active agent, so as to provide an initial rapid increased rate of release of the agent. Dosage forms according to the invention can be administered at least every 8 hours and preferably administered one-a day (every 24 hours) or twice daily (every 12 hours).

Gelatin capsules include soft gelatin capsules or hard gelatin capsules. A soft gelatin capsule is often a one piece hermetically or similarly effectively sealed capsule composed essentially of gelatin which may be plasticized or which may contain other gelatinous material that retains plasticity without becoming brittle. For example, a soft gelatin capsule can be transparent and colorless or pale yellow. Additionally or alternatively, for example, a soft gelatin capsule may have a colorant added. A hard gelatin capsule is often a two piece (cap and body) capsule shell composed of gelatin or other gelatinous material with the appearance of having been or chemically plasticized to the extent of retaining in the unfilled or filled condition a specified shape with a near brittle or brittle physical property. For example, a hard gelatin capsule can be opaque and/or a colorant can be added. A hard gelatin capsule is formed and filled in separation operations. The gelatin capsule fill may be a liquid, semisolid, or solid.

Controlled release or sustained release refers to formulation that provides a longer period of pharmacological response after the administration of a therapeutically active agent that is ordinarily provided after the administration of the immediate release or rapid release formulation of that agent. Controlled release or sustained release formulation which allow the release of a therapeutically active agent or agents in blood levels within a desired therapeutic range and maintains such levels over an extended period of time, such as from at least about 8 hours, such as from about 12 hours to about 24 hours. Controlled release formulations generally contain a controlled release material in order to achieve the controlled or sustained release of the desired agent. A controlled release material can include a continuous matrix, such as an insoluble polymeric matrix or a high viscosity (e.g. non-polymeric) liquid material, wherein a therapeutically active agent is dispersed within and is subsequently released typically by a diffusion-like process of the liquid material, therapeutically active agent through the continuous matrix. Controlled release formulations can also refer to a dosage form comprising a therapeutically active agent that is coated with a controlled release material, so as to permit release of the therapeutically active agent at a sustained rate in an aqueous medium. The controlled release may be a sustained release or delayed/modified release. A controlled-release dosage form as defined in US Pharmacopeia XXII includes extended release dosage forms which allow at least a twofold reduction in dosing frequency as compared to the drug presented as a conventional dosage from and delayed release dosage forms which release the drug at a time other than promptly after administration.

Immediate release generally refers to formulations that allow the release of a therapeutically active agent or agents in blood levels within a desired therapeutic range in a rapid period of time, such as, for example, from about 5 minutes to about 20 minutes. An immediate release formulation can include soluble components, for example, sugars, polymers, surfactants, coatings and other components as described herein.

Therapeutically active agent refers to a substance, including a biologically active substance that is useful for human therapy, veterinary therapy, or for agricultural purposes. Therapy includes prophylactic and therapeutic treatment. Therapeutically active agents include organic molecules that are drugs, peptides, proteins, carbohydrates, monosaccharides, oligosaccharides, polysaccharides, nucleoprotein, mucoprotein, lipoprotein, synthetic polypeptide or protein, small molecules linked to a protein, glycoprotein, steroid, nucleic acid, DNA, cDNA, RNA, nucleotide, nucleoside, oligonucleotides, antisense oligonucleotides, gene, lipid, hormone, and vitamin. Drugs include any substance used as a medicine for the treatment, cure, or prevention of a disease, condition, or disorder. Non-limiting examples of therapeutically active agents include antihistamines, analgesics, anti-inflammatory agents, gastro-intestinals, anti-emetics, anti-epileptics, vasodilators, anti-tussive agents, expectorants, anti-asthmatics, anti-spasmodics, hormones, diuretics, anti-hypertensives, bronchodilators, anti-inflammatory steroids, antivirals, antibiotics, antihemorrhoidals, hypnotics, psychotropics, antidiarrheals, mucolytics, sedatives, decongestants, laxatives, antacids, vitamins, stimulants, and opioids. Among the preferred therapeutically active agents are analgesics, including opioids. A therapeutically active agent includes a first agent that increases the effectiveness of a second agent, for example, by enhancing potency or reducing an adverse effect(s) of the second agent. A therapeutically active agent includes an agent that increases an effect of, acts synergistically with, and/or promotes, potentiates, or enhances an effect of another agent. Such therapeutically active agents are biologically active substances in accordance with the invention. The effect that is increased, promoted, potentiated or enhanced may be, for example, an analgesic effect and the therapeutically active agent may potentiate the analgesic effect of a different therapeutically active agent.

Opioids include compounds or compositions including metabolites as well as conjugates, such as by glucoronidation, sulfation, or acetylation of such compounds or compositions which bind to specific opioid receptors and have agonist (activation) or antagonist (inactivation) effects at these receptors, such as opioid alkaloids, including the agonist morphine as well as morphine-6-glucuronide, oxycodone as well as oxymorphone and noroxycodone, and the antagonist naltrexone and its metabolite, and such as opioid peptides, including enkephalins, dynorphins and endorphins. Opioid receptor agonists or opioid agonists are opioid compounds or compositions including any active metabolite as well as conjugates, such as by glucoronidation, sulfation, or acetylation of such compound or composition that binds to and activates opioid receptors on nociceptive neurons which mediate pain. Such opioid agonists have analgesic activity (with measurable onset, peak, duration and/or total effect and can produce analgesia). Opioid antagonists refer to opioid compounds or compositions including any active metabolite as well as conjugates, such as by glucoronidation, sulfation, or acetylation of such compound or composition that in a sufficient amount attenuates (e.g., antagonizes, blocks, inhibits, prevents or competes with) the action of an opioid agonist.

The controlled release core of the present invention is coated with an immediate release gelatin capsule coating using any of the many gelatin capsule coating processes, such as spray coating, wet gelatin bath dipping, encapsulating, and vacuum holding, for instance. The process of coating an core where the core is, for example, a liquid, tablet, or capsule, with an gelatin coating is also referred to as encasing, enrobing, or encapsulating.

The controlled release core can be in the form of a tablet that is enrobed with the immediate release gelatin capsule coating, wherein the tablet core and the gelatin capsule coating each contain at least one therapeutically active agent, and wherein the immediate release gelatin capsule coating is formed by application of respective layers of elastic gelatin film with the agent to opposite sides of the tablet. This enrobing process without the agent is described, for example, in U.S. Pat. No. 5,146,730, the disclosure of which is incorporated herein by reference. In this process, the applied gelatin layer conforms tightly to the table surface, bonds securely thereto, and the layers are sealed together edge-to-edge at a seal line which extends around the tablet.

The controlled release core can be in the form of a tablet that is enrobed between two sealable gelatin films of the immediate release gelatin capsule coating with the agent according to the invention. An enrobing process without the agent is described, for example, in U.S. Pat. No. 6,482,516, the disclosure of which is incorporated herein by reference. U.S. Pat. No. 6,482,516 describes an enrobement process which uses coacting die techniques wherein tablets or other articles to be enrobed are introduced individually between two sealable gelatin films.

The controlled release core can be in the form of a liquid comprising insoluble particles. This liquid core is encapsulated within the immediate release gelatin capsule with the agent that is in the form of a soft gelatin capsule. An encapsulating process without the agent is described, for example, in U.S. Pat. No. 5,595,758, the disclosure of which is incorporated herein by reference. U.S. Pat. No. 5,595,758 describes a capsule having a soft, flexible gelatin skin and an internal fill which comprises a pharmaceutically acceptable liquid carrier which is compatible with the gelatin coating and which contains small drug-bearing particles which do not dissolve in the liquid.

The immediate release gelatin capsule coating of the present invention comprises at least one therapeutically active agent. Processes of preparing gelatin capsules are described herein. According to the present invention, processes are provided herein to incorporate therapeutically active agents, including drugs or other pharmaceutically acceptable agents, into immediate release gelatin formulations to encapsulate controlled release cores. Soft gelatin capsules (e.g., gel caps) or hard gelatin capsules comprising at least one therapeutically active agent are used as the immediate release gelatin capsule coat according to the invention. Soft gelatin capsules are preferred for incorporating therapeutically active agent(s) according to the invention and preferred manufacturers include Banner Pharmacaps [see e.g., their Softgel, Gelatin Binary System™, and Soflet™ Gelcap products] and Cardinal [see e.g., LIQUI-GELS®, RP SCHERERSOL®, and PULSIN-CAP® technology and products].

The softgel (the currently accepted nomenclature adopted by the SoftGel Association) is a one-piece, hermetically sealed soft gelatin shell containing a liquid, a suspension, or a semi-solid. The most common modern manufacturing process involved in the preparation of softgels is a rotary die process in which a molten mass of a gelatin formulation is fed from a reservoir onto drums to form two spaced sheets or ribbons of gelatin in a semi-molten state. These ribbons are fed around rollers and brought together at a convergent angle into the nip of a pair of roller dies that include opposed die cavities. A liquid or paste medicament or other material to be encapsulated is fed into the wedge-shaped joiner of the ribbons. The gelatin ribbons are continuously conveyed between the dies, with portions of the medicament being trapped between the sheets inside the die cavities. The sheets are then pressed together, and severed around each die so that opposed edges of the sheets flow together to form a continuous gelatin covering around the entrapped medicament. The very soft capsules are then dried to increase the integrity of the capsules, and packaged for later distribution and consumption. See P. Tyle, Specialized Drug Delivery System, Marcel Dekker, Inc. (1990) for a general discussion of softgel manufacturing and production technology, in particular, Chapter 10 by Paul K. Wilkinson and Foo Song Horn, the disclosures of which are incorporated herein by reference.

Various gelatin shell masses may be prepared, depending on the fill properties, climatic conditions, and end use. Typically gelatin formulations include the same basic ingredients, namely, gelatin, a plasticizer such as glycerin, water, and optionally preservatives. Formulations of gelatins are well known. In most cases, the typical rotary die process requires a flowable liquid or fill. The fill may be a single phase liquid active, a mixture of miscible liquids, or a solution or a suspension of solids and liquids. Generally the fill contains glycerin and a medicament. Liquids to be encapsulated in a gelatin shell are also well known. Shell and fill formulations are discussed in Van Hostetler and J. Q. Bellard noted below as well as in “Advances in Softgel Formulation Technology”, M. S. Patel, F. S. S. Morton, and H. Seager, Manufacturing Chemists, July 1989; “Soft Elastic Gelatin Capsules: A Unique Dosage Form”; William R. Ebert, Pharmaceutical Technology, October 1977; “Soft gelatin capsules: a solution to many tableting problems”, H. Seager, Pharmaceutical Technology, September 1985; U.S. Pat. Nos. 4,067,960, 4,198,391, 4,744,988, and 4,780,316, the disclosures of all of which are incorporated herein by reference.

After the rotary die process is used to thereby produce gelatin shells having a medicament fill therein, the resulting capsules are typically washed with an evaporatable solvent. Thereafter, the capsules are typically dried a temperature typically less than 35° C. After the drying process, a large proportion (50-60%) of the water from the gelatin shell has been removed. Recent developments in drying include bypassing the drum drying stage and having the capsules dried in a drying tunnel or room as discussed below.

After the capsules exit the last drying drum, the capsules are typically spread on drying trays. The final drying phase for softgels is typically accomplished by passing the drying trays through drying tunnels or into drying rooms. Stacks of trays are inserted into drying tunnels or drying rooms, in which controlled temperature air (21°-24° C.) and low relative humidity (20%-30%) is continuously circulated. Although additional water may be removed from dry capsules by further heating, for example at 40° C., such a procedure has not been found to be practical or necessary. See, e.g., Van Hostetler and J. Q; Bellard in The Theory and Practice of Industrial Pharmacy, “Capsules”, (1970), Chapter 13 at pages 346-383, and in particular at page 380, the disclosure of which is incorporated herein by reference.

The drying time, for most softgels, is 16-24 hours, but may be slightly longer if the softgels are over 20 minims in size or if the softgels contain a non-oily type liquid base. Evaporation of liquids including ethanol or water can occur during the drying process. Softgels permitted to come to water equilibrium in this controlled environment are considered “dry”. The gelatin fill and shell of such “dry” softgels contain 6-10% water depending on the specific gelatin and fill formula used. After drying, the capsules are typically inspected and finished using varied known techniques.

The immediate release gelatin capsule can be coated with one or more layers of over-coating. Such overcoating can seal and/or protect the gelatin capsule, including sealing and/or protecting the therapeutically active agent(s) in the gelatin capsule and/or on its surface. The agent(s) can migrate into the gelatin capsule layer or unto the gelatin capsule surface during the drying process and would be protected by such an overcoat. Such overcoating can also improve the mechanical strength of the gelatin capsule. Such over-coating may, for example, comprise hydroxypropyl methylcellulose, as described, for instance, in U.S. Pat. No. 4,816,259, the disclosure of which is incorporated herein by reference. U.S. Pat. No. 4,816,259 describes the application of a hydroxypropyl methylcellulose subcoating to the surface of a soft gel to improve mechanical strength of the capsule and to better adhere enteric coating compositions.

Where the controlled release core is in the form of a tablet, the immediate release gelatin capsule can comprise an overcoat of at least one adhesive gelatin film. This adhesive gelatin coating is advantageous in the gelatin capsule drying process because it can become an integral part of the finished product dosage form and not be physically removed without damaging the finished product dosage form or the controlled release core. This feature can be particularly important where the product dosage form to be produced is a tamper-evident medicine formulation. The use of adhesive gelatin coating for tablets is described, for example, in U.S. Pat. No. 5,459,983, the disclosure of which is incorporated herein by reference. Compositions suitable for use as an overcoat of an adhesive gelatin film comprise a plasticizer in an optimal amount. Low ratios of plasticizer to gelatin result in a brittle gelatin film coating whereas high ratios result in a gelatin coating that is flexible and can be peeled from the product dosage form. An example of a composition that is satisfactory for use as an adhesive gelatin coating comprises plasticizer and gelatin in a ratio of about 1:5, respectively.

Any gelatin formulation which can be used successfully in the manufacture of soft or hard gelatin capsules containing flowable materials taking into account matters of technical capability and/or capacity, where the materials include, for example, powder, liquids, compressed solids, or pastes, along with any therapeutically active agent can be used in the immediate release gelatin capsule coating of the present invention. Any pharmaceutically acceptable gelatin suitable for human administration can be employed in the present invention. Gelatin is a coating material used in pharmaceutical formulation. Gelatin is commercially available in many forms, such as acid bone gelatin or lime bone gelatin. Gelatin can be derived by at least partial acid or base hydrolysis of collagen of skin, tendons, ligaments, or bones from a variety of animal sources, such as mammalian or fish, resulting in gelatinous materials with varying bloom strength and compatibility with the therapeutically active agent(s) such as drug, mixed with the gelatin formulation. Bloom refers to the cohesive strength of a gelatinous material. Bloom values are normally determined by measuring the weight in grams required to move a plunger 0.5 inch in diameter, 4 mm into a 6.67% gelatin gel that has been held for 17 hours at 10° C. Conventional soft gelatin capsule have a bloom in the range of from about 150 to about 275. The gelatin in the gelatin capsule may be Type A or B gelatin or a mixture thereof. Limed bone, acid bone, fish, and/or pig skin gelatins may be used.

The immediate release gelatin capsule coating with at least one therapeutically active agent can be in the form of a chewable soft gelatin capsule. Chewable soft gelatin capsules comprise a chewable gelatin encapsulating a liquid fill which are designed to at least partially disperse or dissolve in the user's mouth within a brief period of time after the fill contents have been released, such as within about 60 seconds, so that it can be swallowed. Chewable soft gelatin capsules are described, for example, in U.S. Pat. No. 6,258,380, the disclosure of which is incorporated herein by reference. Capsule formulations compatible for use in chewable soft gelatin capsules are formed of a mixture of a first gelatin having a bloom substantially lower than the bloom of gelatins convention used to form capsules, in combination with a minor percentage of a second gelatin having a bloom within the range of conventional capsule-forming gelatin blooms. A non-limiting example of a capsule formulation comprises: (i) a first gelatin having a bloom of from about 80 to about 100 in an amount of from about 20% to about 30% by weight, (ii) a second gelatin having a bloom of from about 150 to about 275 in an amount of from about 5% to about 29% by weight, (iii) up to about 10% water, (iv) a plasticizer in an amount sufficient to render the capsule flexible, and (v) a moisture retention agent in an amount sufficient to provide capsule integrity. The capsule formulation further comprises at least one therapeutically active agent.

The immediate release gelatin capsule coating can be in the form of a gum acacia substituted soft gelatin capsule. Gum acacia substituted soft gelatin capsules are composed from capsule formulations comprising gum acacia as a gelatin extender. Gum acacia or gum arabic or acacia is a plant exudates collected from the trees of Acacia species. Gum acacia is an arabino-galactan-protein complex composed by weight of from about 17% to about 34% arabinose, from about 32 to about 50% galactose, from about 11 to about 16% rhamnose, from about 13% to about 19% glcuronic acid and from about 1.8% to about 2.5% protein. Capsule formulations comprising gum acacia are described, for example, in U.S. Pat. No. 6,139,999, the disclosure of which is incorporated herein by reference. Gum acacia can replace gelatin, in replacement amounts of from about 5% to about 35% by total weight of gelatin in capsule forming compositions. These compositions may be used in thermally sealed, orally administered capsules manufactured by conventional rotary dies encapsulation machines, without increasing the brittleness of the gelatin shell. Other advantages in formulating softgels with gelatin compositions comprising gum acacia include, for example, shorter drying periods because gum acacia is a film-former whereas gelatin is not, shorter aging times of gel masses to allow for shortening production cycles and increasing throughput, and shorter opening and disintegration times for finished capsules due to the highly cold-water soluble features of gum acacia. An example of a gum acacia substituted softgel composition comprises: (i) a film forming material in an amount ranging from about 30% to about 60% by weight, (ii) a water-dispersible or water-soluble plasticizer in an amount ranging from about 5% to about 35% by weight, (iii) purified water in an amount ranging from about 25% to about 65% by weight, wherein the film forming material comprises gelatin and gum acacia, with gum acacia accounting for from about 0.5% to about 50% by weight of the total amount of the film-forming material, a dried film having from 3% to about 12% by weight of water formed from the composition. The capsule formulation further comprises at least one therapeutically active agent.

The immediate release gelatin capsule coating can be in the form of a softgel that includes a filled portion and a non-filled portion wherein at least one of the filled and non-filled portions has an external surfacing having defined thereon an impressed graphical representation, such as a letter, number, symbol, logo, or the like. Methods for making a softgel that have a filled portion and a non-filled portion whereby upon sealing, a graphical representation is impressed as described, for example, in U.S. Pat. No. 5,827,535, the disclosure of which is incorporated herein by reference.

The immediate release gelatin capsule coating can be in the form of a multiple layer softgel. Multiple layer softgels refer to softgel capsules which comprise a first gelatin layer having a certain thickness and a second layer having another certain thickness wherein the second gelatin layer at least partially surround the first gelatin layer. Such multiple layer softgels are described, for example, in U.S. Pat. No. 6,183,845, the disclosure of which is incorporated herein by reference. An example of a multiple layer softgel is a softgel capsule with content, having opposing ends comprising a first sheet that covers a first end, the first sheet comprising at least a first gelatin layer and a second layer, each layer having a uniform thickness, and a second sheet that covers a second end, the second sheet comprising at least a third gelatin layer, the third layer having a uniform thickness wherein the first and second sheets meet at a seam. At least one of the multiple gelatin layers comprise at least one therapeutically active agent.

The immediate release gelatin capsule coating can be in the form of a one-piece gelatin capsule or shell that includes a plasticizer to control the softness and flexibility of the capsule, water, and optionally other additives, such as flavorants, colorants, opacifiers, etc. Such soft or hard gelatin compositions are described, for example, in U.S. Pat. No. 6,251,426, the disclosure of which is incorporated herein by reference. The softgel capsule may be produced in a known manner with a rotary die process in which a molten mass of a gelatin formulation is fed from a reservoir onto drums to form two spaced sheets or ribbons of gelatin in a semi-molten state. These ribbons are fed around rollers and brought together at a convergent angle into the nip of a pair of roller dies that include opposed die cavities.

Additional examples of shell formulation that can be used in soft gelatin capsules are described, for example in Van Hosteteler and J. Q. Bellard in “Advances in Softgel Formulation Technology”, M. S. Oatel F. S. S. Morton, and H. Seager, Manufacturing Chemists, July 1989; “Soft Elastic Gelatin Capsules: A Unique Dosage Form”, William R. Ebert, Pharmaceutical Technology, October 1977; “Soft gelatin capsules: a solution to many tableting problems”, H. Seager, Pharmaceutical Technology, September 1985; U.S. Pat. Nos. 3,959,540, 4,198,391, 4,744,988, 4,780,316, 5,200,191, 5,380,534, 5,422,160, 5,484,598, 5,505,961, 5,569,466, 5,595,758, 5,624,681, 5,682,733, 5,735,105, 5,750,145, 5,817,323, 5,827,535, 5,891,470, 5,985,321, 6,096,338, 6,120,806, 6,183,845, 6,193,999, 6,214,376, 6,251,426, 6,285,380, 6,288,894, 6,387,400, the disclosures of all of which are incorporated herein by reference. Gelatin shell formulations in contrast to prior indices, comprise at least one therapeutically active agent.

Various gelatin capsule formulations may be used to encapsulate the controlled released core. For example, suitable capsule formulation may include gelatin in an amount of about 35% to about 50% by weight, a plasticizer in an amount of from about 20% to about 40%, and water in an amount of from about 25% to about 50%. The exact weight percentage of gelatin to be used in the immediate release gelatin capsule coating can be readily optimized by routine experimentation to achieve a gelatinous composition of desired bloom strength. Since all forms of gelatin are water soluble and comprise of hygroscopic protein, the water content of gelatin formulations to be used in the immediate release immediate release gelatin capsule coating can vary; however, the water content of the gelatinous composition can also be readily optimized. In addition, plasticizers, additives, colorants, preservants, and protectants may be added to the gelatinous composition to enhance the aesthetic and mechanical features (i.e. softness and flexibility) of the gelatin capsule. Again, the type and amount of plasticizers, additives, colorants, preservants, and protectants in such gelatin formulations to be used in the immediate release immediate release gelatin capsule coating can be readily optimized to achieve the desired effect. Examples of plasticizers that can be used include, for instance, sorbitol, sorbitol with sorbitan, (as described, for example, in U.S. Pat. No. 4,744,988, the disclosure of which is incorporated herein by reference), malitol, (as described, for example, in U.S. Pat. No. 5,569,461 the disclosure of which is incorporated herein by reference), glycerol, xylitol, polyglycerol, glucose, fructose, or a mixture thereof. Also, surfactants and drying agents may be added to the gelatin formulation of the present invention, such as when the gelatin formulation is to be used in a spray coating process as described in U.S. Pat. No. 6,077,540, for example. Surfactants may act to complex gelatin proteins thereby restraining the adhesive character of gelatin. Non-limiting examples of surfactants include stearoyl lactylate, calcium steroyl lactylate, and glyceryl monosterarate. Drying agents may act to desolvate gelatin and shorten the drying time of the gelatin coating. Non-limiting examples of drying agents include magnesium aluminum silicate and sodium, magnesium and potassium sulfate, and hydrophilic clays. For gelatinous compositions comprising hydrophilic clays and magnesium aluminum silicate, these two substances have been described as suspending agents and may play a dual role in these compositions. Capsule formulations may also contain taste modifiers, coloring agents, and moisture retaining agent. Examples of taste modifiers include, for instance, non-reducing sugars, such as xylitol, malitol, or Lycasin® manufactured by Roquette America Inc. of Keokuk, Iowa and may comprise up to about 5% by weight of the gelatin capsule composition. Examples of moisture retaining agents include, for instance, celluloses, cellulose derivatives, starches, starch derivatives, vegetable gums, non-hygroscopic mono- and di-oligosaccharides, starch acetates, starch derivatives, potato unbleached starch acetate (as described, for example, in U.S. Pat. No. 5,817,323, the disclosure of which is incorporated herein by reference), and silicon dioxide. In determining the exact gelatinous composition to be used in formulating the immediate release gelatin capsule coating, factors such as ease of handling, cost, and adaptability to subsequent processes, for example, are considered.

For consumer acceptability, immediate release gelatin capsules in the form of a softgel capsule should be of size that is easily swallowed. Generally, the fill size of the capsule can be less than 60 mg, such as about 500 mg or less, for the capsule to be of an acceptably small dimension, although other sizes are possible. The controlled release core can comprise at least one surfactant, such as polyethylene glycol or polyvinylpyrrolidone, to accommodate a particular fill volume (as described, for example, in U.S. Pat. No. 6,387,400, the disclosure of which is incorporated herein by reference). Also, the controlled release core may be free of water and other ingredients that increase fill volume. The controlled release core can also comprise ethanol and/or at least one partial glyceride of fatty acids for stable preparation. Such fill compositions comprise ethanol in an amount of from about 5% to about 50% by weight and at least one partial glyceride of fatty acids having from about 6 to about 18 carbon atoms in an amount of from about 20% to about 95% by weight. These ethanol containing fill compositions are described, for example, in U.S. Pat. No. 4,888,239, the disclosure of which is incorporated herein by reference.

The immediate release gelatin capsule coating composition may be manufactured by an improved process comprising subjecting “dry” softgels to a subsequent stress relieving step such that the volume and number of defects such as dimples and bubbles existing in the softgels prior to the stress relieving step can be substantially reduced. In addition, the stress-relieving step reduces dimensional standard deviation thereby resulting in more dimensionally uniform batches of softgels. The stress relieving step is described, for example, in U.S. Pat. No. 5,200,191, the disclosure of which is incorporated herein by reference. The stress relieving step comprises subjecting the “dry” capsules to a subsequent heating step at a heightened temperature, such as from about 32° C. to about 42° C., and relative humidity, such as from about 35% to about 60% relative humidity.

The immediate release gelatin capsule coating of the present invention comprises at least one of any therapeutically active agent, including, for example, an opioid agonist and/or an opioid antagonist. In preferred embodiments, the immediate release gelatin capsule coating of the present invention comprises at least one opioid agonist and/or at least one opioid antagonist. Representative opioid agonists include at least one of the following: alfentanil, allylprodine, alphaprodine, anileridine, apomorphine, apocodeine, benzylmorphine, bezitramide, buprenorphine, butorphanol, clonitazene, codeine, cyclazocine, cyclorphen, cyprenorphine, desomorphine, dextromoramide, dezocine, diampromide, dihydrocodeine, dihydromorphine, dimenoxadol, dimepheptanol, dimethylthiambutene, dioxyaphetyl butyrate, dipipanone, eptazocine, ethoheptazine, ethylmethylthiambutene, ethylmorphine, etonitazene, fentanyl, heroin, hydrocodone, hydroxymethylmorphinan, hydromorphone, hydroxypethidine, isomethadone, ketobemidone, levallorphan, levorphanol, levophenacylmorphan, lofentanil, meperidine, meptazinol, metazocine, methadone, methylmorphine, metopon, morphine, myrophine, nalbuphine, narceine, nicomorphine, norlevorphanol, normethadone, nalorphine, normorphine, norpipanone, ohmefentanyl, opium, oxycodone, oxymorphone, papavereturn, pentazocine, phenadoxone, phenomorphan, phenazocine, phenoperidine, pholcodine, piminodine, piritramide, propheptazine, promedol, profadol, properidine, propiram, propoxyphene, remifentanyl, sufentanyl, tramadol, tilidine, salts thereof, mixtures of any of the foregoing, mixed mu-agonists/antagonists, mu-antagonist combinations, or others known in the art. Some of the opioid agonists and/or antagonists disclosed herein may contain one or more asymmetric centers and may thus give rise to enantiomers, diastereomer, and other stereoisomeric forms. The present invention is also meant to encompass all such possible forms as well as their racemic and resolved forms and mixtures thereof. When the compounds described herein contain olefinic double bond or other centers of geometric asymmetry, and unless specified otherwise, it is intended to include both E and Z geometric isomers. All tautomers are intended to be encompassed by the present invention as well.

Representative opioid antagonists include at least one of the following: naltrexone (marketed in 50 mg dosage forms from Du Pont Pharma as ReVia® or Trexan®), naloxone (marketed as Narcan®, NALOXONE/PENTAZOCINE® from Pharma Pac), nalmefene, methylnaltrexone, naloxone methiodide, nalorphine, naloxonazine, nalide, naltrexone, nalbuphine, nalorphine dinicotinate, naltrindole (NTI), naltrindole isothiocyanate, (NTII), naltriben (NTB), nor-binaltorphimine (nor-BNI), b-funaltrexamine (b-FNA), BNTX, cyprodime, ICI-174,864, LY117413, MR2266, or an opioid antagonist having the same pentacyclic nucleus as nalmefene, naltrexone, buprenorphine, levorphanol, meptazinol, pentazocine, dezocine, or their pharmacologically effective esters or salts. Commercial formulations, including commercial oral dose forms, currently used to administer an opioid agonist or opioid antagonist can be modified as described and used to provide oral dosage forms according to the present invention. In particular, commercial controlled release oral dosage forms of opioid agonists, including, for example, oxycodone, hydrocodone, or morphine that are tablets or capsules may be enrobed with an immediate release gelatin capsule coating comprising the opioid agonist alone or in combination with an opioid antagonist. Commercial oral dose forms of opioid agonists for human administration include: codeine, dihydrocodeine (e.g., SYNALGOS-DC® from Wyeth-Ayerst Pharmaceuticals), fentanyl, hydrocodone (e.g., NORCET® from Abara; DOLOREX FORTE® from A.G. Marin; VICODIN TUSS® from Allscripts; HY-PHEN® from Ascher, HYCODAN® and ZYDONE® from Endo Pharmaceuticals; BANCAP HC®, LORCET 10/650®, LORCET PLUS®, and LORCET-HD® from Forest Pharm; VANACET® from GM Pharm; VICODIN®, VICODIN ES®, VICODIN HP®, VICODIN TUSS EXPECTORANT®, and VICOPROFEN® from Knoll Pharma; ANEXSIA®, HYDROCET®, and LORCET-HD from Mallinckrodt; HYCOMED® from Med-Tek; CO-GESIC® from Schwarz Pharma; CETAPLUS® from Seatrace; LORTAB® and VICON FORTE® from UCB Pharma; NORCO® from Watson Laboratories; ALLAY® from Zenith Goldline), hydromorphone (e.g., DILAUDID® from Knoll), levorphanol (e.g., LEVO-DROMORAN® from ICN Pharmaceuticals), meperidine (e.g., DEMEROL® from Sanofi Pharmaceuticals), methadone (e.g., METHADOSE® from Mallinckrodt; and DOLOPHINE® HCl from Roxane Laboratories), morphine (e.g.,® from Allscripts; KADIAN® from Faulding Laboratories; AVINZA™ from Elan/Ligand; MS CONTIN® from Purdue Frederick; ORAMORPH® SR from Roxane; RMS® from Upsher-Smith), nalbuphine (e.g., NUBAIN® from Endo Labs), oxycodone (e.g., PERCOCET®, PERCODAN®, and PERCOLONE® from Endo; OXYCET® from Mallinckrodt; TYLOX® from Ortho-McNeil Pharmaceutical; OXYCONTIN® and OXYFAST® from Purdue Pharma; ROXICODONE®, ROXILOX®, ROXICODONE-INTENSOL®, ROXANOL®, ROXANOL-100®, ROXANOL-T®, and ROXICET® from Roxane), oxymorphone (e.g., NUMORPHANHCL® from Endo Labs), pentazocine (e.g., TALACEN® and TALWIN® from Sanofi Pharmaceuticals), propoxyphene (e.g., PROPDXYPHENE HYDROCHLORIDE® from Allscripts; PC-CAP® from Alra; PRONAP® from DHS Inc; DARVOCET-N®, DAVRON®, DAVRON-N®, DAVRON COMPOUND-65® from Eli Lilly & Co.; DOLENE® from Lederle; PROPDXYPHENE HCL COMPOUND® from Major, PROPDXYPHENE COMPOUND-65® from Mylan; PROPDXYPHENE COMPOUND® from PD-RX Pharm, Phys Total Care, and Southwood; PROPDXYPHENE COMPOUND-65® and PROPDXACET-N® from Quality Care; WYGESIC® from Wyeth-Ayerst), and tramadol (e.g., ULTRAM® from Ortho-McNeil Pharmaceutical). Commercial liquid dose forms of opioid agonists for human administration include: hydrocodone (e.g., HYDROPHANE® from Halsey), hydromorphone (e.g., DILAUDID® from Knoll), meperidine (e.g., DEMEROL® from Sanofi), methadone (e.g., DOLOPHINE® from Roxane), oxycodone (e.g., HYCOMINE® from Knoll; ROXILOX® from Roxane), and propoxyphene (e.g., DARVON-N® from Eli Lilly). Commercial parenteral dose forms for human administration include: alfentanil (e.g., ALFENTA® from Akorn and Taylor Pharm), alfenantil hydrochloride from Abbott Hosp, buprenorphine and buprenorphine/haloxone (e.g., BUPRENEX® and SUBUTEX/SUBOXONE®, respectively from Reckitt & Colman Pharmaceuticals), buprenophine hydrochloride from A-A Spectrum, butorphanol (e.g., STADOL® from Apothecon), codeine (e.g., DURAGANEDIN NR® from Duramed), dextrose morphine from Abbott Hosp, dezocine (e.g., DALGAN® from Astrazeneca), fentanyl (e.g., DURAGESIC® from Janssen), hydrocodone (e.g., DURATUSS HD® from UGB Pharma, HYDROCODONE ES® from Quality Care), hydromorphone (e.g., DILAUDID®, DILAUDID COUGH®, DILAUDID-HP® from Knoll Pharma), levallorphan (e.g., LORFAN® from Roche), levorphanol (e.g., LEVO-DROMORAN® from ICN), meperidine (e.g., DEMEROL® from Sanofi), methadone (e.g., DOLOPHINE® HCI and METHADONE HCL INTENSOL® from Roxane, METHADOSE® from Mallinckrodt Pharma), morphine (e.g, ASTRAMORPH® from Astrazeneca; DURAMORPH® and INFUMORPH® from Elkins-Sinn; KADIAN® from Faulding Labs, MS CONTIN® and MSIR® from Purdue Frederick), oxymorphone (e.g., NUMORPHAN® from Endo), nalburphine (e.g., NUBAIN® from Endo Pharmaceutical), and pentazocine (e.g., TALWIN® from Abbott). Commercial suppository dose forms of opioid agonists for human administration include oxymorphone (e.g., NUMORPHAN® from Endo).

According to the invention, the therapeutically active agent of the immediate release gelatin capsule coating can be an opioid agonist or metabolite, as well as conjugate thereof, such as morphine, tramadol, oxycodone, hydrocodone, oxymorphone, or hydromorphone. The therapeutically active agent of the immediate release gelatin capsule coating can be an opioid antagonist, such as naltrexone or nalmefene. Finally, the therapeutically active agent of the immediate release gelatin capsule coating can be a combination of an opioid agonist and an opioid antagonist, such as naltrexone and oxycodone, respectively.

The amount of a therapeutically active agent included in the immediate release gelatin capsule of dosage forms according to the invention is any pharmaceutically acceptable amount that is sufficient to achieve an increase in the rate of release of the therapeutically active agent from the oral dosage form via the immediate release gelatin capsule comprising the therapeutically active agent, as compared to the rate of release from a controlled release core of the therapeutically active agent. The amount of the therapeutically active agent in the immediate release gelatin capsule of dosage form according to the invention can also or alternatively be an amount sufficient to achieve a rapid release of the therapeutically active agent from the dosage form in the GI tract (e.g., from about less than about 1 minute to less than about 1.5 hours. The amount of the therapeutically active agent in the immediate release gelatin capsule of dosage forms according to the invention can also or alternatively depend upon the desired release profile and the concentration required for a desired biological effect. Additional or alternative factors used to determine the amount of the therapeutically active agent in the immediate release gelatin capsule include, for example, distribution, absorption, and elimination rates of the therapeutically active agent.

According to the invention, the therapeutically active agent in the immediate release gelatin capsule is an opioid agonist that is present in a human subject in an analgesic or subanalgesic amount, including, for example, a non-analgesic amount. Alternatively or additionally, the immediate release gelatin capsule includes an opioid antagonist. When the therapeutically active agent in the immediate release gelatin capsule is a combination of an opioid agonist and an opioid antagonist, the opioid agonist can be present in a human subject in an analgesic or subanalgesic amount, including, for example, a non-analgesic amount.

An analgesic amount includes an amount of the opioid agonist which causes analgesia in subject administered the opioid agonist alone, and also includes standard doses of the opioid agonist which are typically administered to cause analgesia (e.g. mg doses). A subanalgesic amount includes an amount which does not cause analgesia in a subject administered with the opioid agonist alone, but when used in combination with the opioid antagonist, results in analgesia. A non-analgesic amount includes an amount which does not cause analgesia when administered to a subject while an “anti-analgesic” amount is an amount which causes algesia (i.e. pain) when administered to a subject. The amount of the opioid antagonist may be an amount effective to enhance analgesic potency of and/or attenuate one or more adverse side effects of an opioid agonist, including, for example, nausea, vomiting, headache, dizziness, somnolence, pruritus, tolerance, withdrawal, dependence, and/or addiction. Such adverse side effects can include any known undesirable side effect of opioid agonists. The amount of the opioid antagonist may be less than an effective antagonistic amount or an ineffective antagonistic amount, yet still provide some or all of the foregoing benefits. The optimum amounts of the opioid antagonist administered alone or in combination with an opioid agonist or other therapeutic agent will, of course, depend upon the particular agonist and antagonist used, the excipients chosen, the route of administration, and/or the pharmacokinetic properties of the patient being treated.

Oral dosage forms comprising opioids, including an opioid agonist alone or in combination with an opioid antagonist are described, for example, in WO 01/85257 A2, WO 01/58447 A1, U.S. Pat. No. 6,475,494, U.S. Pat. No. 6,375,957, and U.S. Patent Application Publication 2002/001012, the disclosures of which are incorporated herein by reference. These patents and patent publications do not disclose or suggest the application of an immediate release gelatin capsule coating comprising a therapeutically active agent as disclosed herein. Moreover, these patents and patent publications do not disclose or suggest the combination of a controlled release core encapsulated with an immediate release gelatin capsule each comprising at least one therapeutically active agent.

In one aspect of the invention, the therapeutically active agent of the immediate release gelatin capsule is an opioid antagonist, such as naltrexone or nalmefene, and is provided in an amount of about 0.000001 to about 1.0 mg, alternatively less than about 1.0 mg, alternatively less than about 0.5 mg. Preferred ranges of opioid antagonists also include: from about 0.000001 mg to less than 1.0 mg; from about 0.00001 mg to less than 1.0 mg, from about 0.0001 mg to less than 1.0 mg; from about 0.001 mg to less than 1.0 mg, from about 0.01 mg to less than 1.0 mg; or from about 0.1 mg to less than 1.0 mg. Additional preferred ranges of opioid antagonists include: from about 0.000001 mg to less than 1.0 mg; from about 0.00001 mg to less than 1.0 mg, from about 0.0001 mg to about 0.1 mg; from about 0.001 mg to about 0.1 mg; from about 0.01 mg. to about 0.1 mg; from about 0.001 mg to about 0.1 mg; from about 0.001 mg to about 0.01 mg; or from about 0.01 mg to about 0.1 mg. Further preferred ranges of opioid antagonists include: from about 0.000001 mg to less than 1.0 mg; from about 0.00001 mg to less than 1.0 mg, from at least about 0.0001 to less than about 0.5 mg; from at least about 0.01 to less than about 0.5 mg; or from at least about 0.1 to less than about 0.5 mg. Alternatively, the maximum amount of opioid antagonist in the immediate release gelatin capsule is 1 mg. Alternatively, the maximum amount of opioid antagonist in the immediate release gelatin capsule is less than 0.5 mg. The minimum amount of opioid antagonist in the immediate release gelatin capsule is 0.000001 mg. Any minimum amount and any maximum amount of antagonist in the immediate release gelatin capsule, as specified above, may be combined to define a range of amounts in increments of 0.000001 or 0.00001 or 0.0001 or 0.001 or 0.01 or 0.1, providing that the minimum selected is equal to or less than the maximum selected. In an embodiment of the invention, the amount of antagonist in the immediate release gelatin capsule is less than an effective amount to antagonize an exogenous or endogenous opioid agonist, but such an amount may include an amount that enhances the potency and/or attenuates an adverse effect of the agonist, including, for example, nausea, vomiting, headache, dizziness, somnolence, pruritus, tolerance, withdrawal, dependence, and/or addiction.

In another aspect of the invention, the therapeutically active agent of the immediate release gelatin capsule is an opioid agonist alone, such as oxycodone, and is provided in an amount from about 0.0025 mg to about 60 mg.

In yet another aspect of the invention, the therapeutically active agent in the immediate release gelatin capsule is a combination of an opioid agonist, such as oxycodone, oxymorphone, hydrocodone, hydromorphone, morphine, or tramadol and an opioid antagonist, such as naltrexone or nalmefene. Preferably, the opioid antagonist is present in an amount of about 0.000001 to about 1.0 mg, alternatively less than about 1.0 mg, alternatively less than about 0.5 mg, and the opioid agonist is present in an amount from about 0.0025 to about 60 mg. Optionally, the opioid agonist and opioid antagonist are released concurrently over a period of less than about 1.5 hours, including for example, over a period of about 5 minutes to about 20 minutes.

The controlled release core of the present invention comprises at least one therapeutically active agent and at least one controlled release material. Any controlled release material that does not substantially interfere with the solubility of the therapeutically active agent can be used in the controlled release core of the present invention. The controlled release core of the present invention may be in any pharmaceutically acceptable dosage form, preferably capsules, tablets, or caplets.

Controlled release materials can be at least partially hydrophobic in nature. In some controlled release formulations, a drug-containing particle is coated with or is dispersed within a controlled release material that is a continuous matrix, such as a polymeric matrix. The coating layer or matrix can comprise insoluble materials and upon diffusion of the soluble drug through the coating layer or matrix by means of resistance, the drug is released in a controlled fashion. Various formulations of controlled release material(s) and soluble drugs) have been described. Controlled release materials are described, for example, in U.S. Pat. No. 6,387,404, U.S. Pat. No. 5,747,058, U.S. Pat. No. 6,413,536, U.S. Pat. No. 5,968,542, WO 01/58447, U.S. Publication No. US 2002/0010127A1, the disclosures of all of which are incorporated herein by reference.

Controlled release materials useful in dosage forms and formulations according to the invention can include at least one hydrophobic and/or at least one hydrophilic material. Hydrophobic materials that are useful include water-insoluble materials with more or less pronounced hydrophilic and/or hydrophobic trends. Any pharmaceutically acceptable hydrophobic material or hydrophilic material which is capable of imparting controlled release of a therapeutically active agent, including, for example, an opioid agonist alone or in combination with an opioid antagonist may be used in accordance with the present invention. Hydrophobic materials that may be used include those having a melting point from about 30° C. to about 200° C., such as from about 45° C. to about 90° C.

The controlled release material can be at least one type of hydrophilic alkylcellulosic material such as hydroxyalkylcellulose or hydroxypropylmethylcellulose.

The controlled release material can be at least one acrylic polymer. The acrylic polymer may be cationic, anionic, or non-ionic polymers and may be acrylates an/or methacrylates, formed of methacrylic acid or methacrylic acid esters. Examples of suitable acrylic polymer include, but are not limited to, acrylic acid and methacrylic acid copolymers, methyl methacrylate copolymers, ethoxyethyl methacrylates, cyanoethyl methacrylate, methyacryacylic acid copolymers, and aminoalkyl methacrylate copolymers. The acrylic polymer can be one or more ammonia methacrylate copolymers. Ammonia methacrylate copolymers are and can be described as fully polymerized copolymers of acrylic and methacrylic acid esters with a low content of quaternary ammonium groups. Additional examples include, but are not limited to, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In order to obtain a desirable dissolution profile, it may be necessary to incorporate two or more ammonia methacrylate copolymers having differing physical properties, such as different molar ratios of the quaternary ammonium groups to the neutral (meth)acrylic esters.

The controlled release material can be a mixture of two acrylic resin lacquers. Compositions comprising a mixture of two acrylic resin lacquers can be used as controlled release coatings. Some commercially available acrylic resin lacquers are from Rohm Pharma under the Tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents.

Eudragit® RL/RS dispersions may be mixed together in any desired ratio in order to ultimately obtain a controlled release formulation having a desirable dissolution profile. Desirable controlled release formulations may be obtained, for instance, from a retardant coating derived from 100% Eudragit® RL, 50% Eudragit® RL and 50% Eudragit® RS, and 10% Eudragit® RL: 90% Eudragit® RS. Other acrylic polymers may also be used, such as, for example, Eudragit® L.

The controlled release core according to the present invention can be formulated so as to not exhibit a significant fed/fast effect. No fed/fast effect refers to pharmacokinetic parameters, such as blood plasma concentration of drug, that exhibit less than a 20% difference in formulations that are administered to patients on an empty stomach versus administration to patients who have ingested a high-fat meal, as defined by the U.S.F.D.A. Sustained release formulations that do not exhibit a food effect are described, for example, in U.S. Application Nos. 2001/0031278 A1 and 2002/0102303 and in WO 97/45091, the disclosures of which are incorporated herein by reference. U.S. Application Nos. 2001/0031278 A1 and 2002/0102303 and WO 97/45091 describe the preparation of sustained release oxycodone formulation which do not exhibit a significant fed/fast effect by utilizing a carrier which preferentially causes the formulation to release the oxycodone in fluids having a relatively lower (acidic) pH. Non-limiting examples of a controlled release formulation that does not exhibit a significant fed/fast effect include a composition comprising Eudragit RSPO in an amount of approximately 48.75% by weight, Eudragit L-100 in an amount of approximately 3.75% by weight, and stearic acid in an amount of approximately 22.5% by weight. Additional non-limiting examples of a controlled release formulation that does not exhibit a significant fed/fast effect include a composition comprising Eudragit R30SD (solid) in an amount of approximately 10.8% by weight, spray dried lactose in an amount of approximately 27.1% by weight, PVP in an amount of approximately 3.9% by weight, triacetin in an amount of approximately 1.5% by weight, stearyl alcohol in an amount of approximately 19.2% by weight, talc in an amount of approximately 1.9% by weight, and magnesium stearate in an amount of approximately 0.9% by weight.

The core can be coated with at least one controlled release material in an aqueous dispersion comprising at least one hydrophobic material and further comprising an effective amount of at least one plasticizer to improve the physical properties of the controlled release coating. For example, because ethylcellulose has a relatively high glass transition temperature and does not form flexible films under normal coating conditions, it is preferable to incorporate a plasticizer into an ethylcellulose coating containing sustained release coating before using the same as a coating material. Generally, the amount of plasticizer included in a coating solution is based on the concentration of the film-former, e.g., most often from about 1 to about 50 percent by weight of the film-former. The concentration of the plasticizer, however, can only be properly determined after routine experimentation with the particular coating solution and its intended method of application. Examples of suitable plasticizers for ethylcellulose include, but are not limited to, water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate can be used as a plasticizer for the aqueous dispersions of ethyl cellulose of the present invention. Examples of suitable plasticizers for the acrylic polymers include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films, such as Eudragit® RL/RS lacquer solutions, include polyethylene glycols, propylene glycol, diethyl phthalate, castor oil, and triacetin. Triethyl citrate can be used as a plasticizer for the aqueous dispersions of ethyl cellulose of the present invention. The addition of a small amount of talc reduces the tendency of aqueous dispersions to stick during processing, and may act as a polishing agent.

The release profile of the controlled release core can be altered, for example, by altering the manner in which the plasticizer is added to the hydrophobic material, by varying the amount of plasticizer relative to hydrophobic material, by the inclusion of additional ingredients or excipients, and/or by altering the method of manufacture. Further modifications to the release profile may also be implemented, for example, by increasing or decreasing the thickness of the retardant coating.

The controlled release core can further include a colorant to provide elegance and product distinction. Color may be added to the solution of the therapeutically active agent instead. Any suitable method of providing color to controlled release formulations may be used. Suitable ingredients for providing color to the formulation when an aqueous dispersion of an acrylic polymer is used include titanium dioxide and color pigments, such as iron oxide pigments. The incorporation of pigments, may, however, increase the retard effect of the coating.

The release of the therapeutically active agent from the controlled release formulation according to the present invention can be further influenced, i.e., adjusted to a desired release rate, by the addition of at least one release-modifying agents, or by providing one or more passageways through the coating. The release-modifying agents which function as pore-formers may be organic or inorganic, and include materials that can be dissolved, extracted, or leached from the coating in the environment of use. The pore-formers may comprise one or more hydrophilic materials such as hydroxypropylmethylcellulose. The release-modifying agent may also comprise a semi-permeable polymer. The release-modifying agent can be selected from hydroxypropylmethylcellulose, lactose, or metal stearates. The controlled release coatings can also include erosion-promoting agents such as starch and gums.

The controlled release material can be at least one material useful for making microporous lamina in the environment of use, such as polycarbonates comprised of linear polyesters of carbonic acid in which carbonate groups reoccur in the polymer chain.

The controlled release material comprises at least one passageway, orifice, or the like. The passageway may be formed by such methods as those disclosed in U.S. Pat. Nos. 3,845,770, 3,916,889, 4,063,064, and 4,088,864, the disclosures of all of which are hereby incorporated by reference. The passageway can have any shape such as round, triangular, square, elliptical, irregular, etc.

The controlled release material can be at least one natural or synthetic wax, fatty alcohol (such as lauryl, myristyl, stearyl, cetyl or preferably cetostearyl alcohol), fatty acid, including but not limited to fatty acid ester, fatty acid glyceride (mono-, di-, and tri-glycerides), hydrogenated fat, hydrocarbon, normal wax, stearic aid, stearyl alcohol, or hydrophobic and/or hydrophilic material having hydrocarbon backbones. Suitable waxes include, for example, beeswax, glycowax, castor wax and carnauba wax. A wax-like substance includes any material which is normally solid at room temperature and has a melting point of from about 30° C. to about 100° C.

The controlled release material can be at least one water-insoluble wax-like thermoplastic substance that is optionally mixed with at least one less hydrophobic wax-like thermoplastic substance. In order to achieve sustained release, the individual wax-like substances in the controlled release material should be substantially non-degradable and insoluble in gastrointestinal fluids during the initial release phases. Useful water-insoluble wax-like substances may be those with a water-solubility that is lower than about 1:5,000 (w/w).

The controlled release material can be at least one digestible, long chain (C₈-C₅₀, such as C₁₂-C₄₀), substituted or unsubstituted hydrocarbon, such as a fatty acid, fatty alcohol, glyceryl ester of at least one fatty acid, mineral oil, or vegetable oil. Hydrocarbons having a melting point of from about 25° C. to about 90° C. may be used in the invention. Fatty (aliphatic) alcohols may be used as a long chain hydrocarbon material.

The controlled release material can be at least one hydroxyalkyl cellulose or acrylic resin and at least one aliphatic alcohol or polyalkylene glycol in a ratio of from about 1:2 to about 1:4, respectively, such as a ratio of from about 1:3 2 to about 1:4, respectively. The at least one polyalkylene glycol may be, for example, polypropylene glycol or polyethylene glycol. The number average molecular weight of the at least one polyalkylene glycol can range from about 1,000 to about 15,000, such as from about 1,500 to about 12,000.

The controlled release material can be at least one of the following: stearate esters, such as those of propylene glycol, glyceryl, diethylaminoethyl, and glycol, stearate amides and other long-chain fatty acid amides, such as N,N′-ethylene disteramide, steramide MEA and DEA, ethylene bisteramide, cocoamine oxide, long chain fatty alcohols, such as cetyl alcohol and steryl alcohol, long chain esters such as myristyl myristate, beheny erucate, glyceryl phosphates, and acetylated sucrose distearate.

The controlled release material can be at least one of the following: methacrylic ester copolymers, poly(ethylacrylate, methyl methacrylate), poly(ethyl acrylate, methyl methacrylate, trimethylamminoethyl methacrylate chloride), polymethyl methacrylate-methacrylic acid copolymers, cellulose acetate, ethylcellulose, cellulose acetate phthalate, and hydroxypropyl methylcellulose phthalate.

The controlled release material can be at least one of the following: polyamides, polycarbonates polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydride, poly(ortho)esters, poly(butic acid), poly(valeric acid), poly (lactide-co-caprolactone), polysaccharides, proteins, polyhyaluronic acids, polycyanoacrylates, and blends, mixtures, or copolymers thereof.

The controlled release material can be at least one type of alkylcellulosic polymer, such as ethylcellulose, although the artisan will appreciate combination, as all or part of the controlled release materials presented herein. One commercially available aqueous dispersion of ethylcellulose is Aquacoat® (FMC Corp., Philadelphia, Pa., U.S.A.). Aquacoat® is prepared by dissolving the ethylcellulose in a that other cellulose and/or alkylcellulose polymers may be readily employed, singly or in any water-immiscible organic solvent and then emulsifying the same in water in the presence of a surfactant and a stabilizer. Another aqueous dispersion of ethylcellulose is commercially available as Surelease® (Colorcon, Inc., West Point, Pa., U.S.A.). Surelease® is prepared by incorporating plasticizer into the aqueous dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion, which can be directly applied.

The core of dosage forms according to the invention comprises a therapeutically active agent that is dispersed within at least one controlled release material. The core of dosage forms according to the invention comprises a therapeutically active agent that is dispersed within a matrix comprising at least one controlled release material. The core of dosage forms according to the invention comprises a therapeutically active agent that is dispersed within a matrix that is coated with at least one controlled release material. The controlled core of dosage forms according to the invention comprises the opioid agonist and optionally, opioid antagonist, which is coated additionally or alternatively with a controlled release material. The controlled release coating or controlled release matrix of the core comprises at least one controlled release material that facilitates in vitro dissolution rates of at least one therapeutically active agent within the preferred ranges disclosed herein.

Materials suitable for inclusion in a controlled release matrix will depend on the particular method used to form the matrix. For example, the controlled release matrix may comprise at least one hydrophilic and/or hydrophobic material, such as gum, cellulose ether, acrylic resin, and protein derived material. The controlled release matrix may comprise of a combination of two or more hydrophobic controlled release materials. Controlled release matrices may also comprise at least one digestible, long chain (C₈-C₅₀, such as C₁₂-C₄₀), substituted or unsubstituted hydrocarbon, such as fatty acid, fatty alcohol, glyceryl ester of fatty acids, mineral and vegetable oil, and wax, stearyl alcohol, and polyalkylene glycol. Of these polymers, acrylic polymers, especially Eudragit® RSPO—the cellulose ethers, especially hydroxyalkylcelluloses and carboxyalkylcelluloses, can be used. The controlled release matrix may comprise at least one hydrophilic or hydrophobic material in an amount ranging from about 1% to about 80% (by weight). In an embodiment where the controlled release matrix comprises at least one hydrocarbon, such as a long chain hydrocarbon or fatty (aliphatic) alcohol, the hydrocarbon can have a melting point of from about 25° C. to about 90° C. and can be present in an amount up to 60% (by weight). In certain embodiments, the controlled release matrix comprises at least one polyalkylene glycol in an amount up to 60% (by weight).

An example of a suitable matrix comprises at least one water-soluble hydroxyalkyl cellulose, at least one C₁₂-C₃₆, such as C₁₄-C₂₂, aliphatic alcohol and, optionally, at least one polyalkylene glycol. The at least one hydroxyalkyl cellulose can be a hydroxy (C₁ to C₆) alkyl cellulose, such as hydroxypropylcellulose, hydroxypropylmethylcellulose, or hydroxyethylcellulose. The amount of the at least one hydroxyalkylcellulose in the invention can be determined, inter alia, by the precise rate of release required for a therapeutically active agent, such as an opioid. The at least one aliphatic alcohol can be, for example, lauryl alcohol, myristyl alcohol, or stearyl alcohol. The at least one aliphatic alcohol can be cetyl alcohol or cetostearyl alcohol. The amount of the at least one aliphatic alcohol can be determined, inter alia, by the precise rate of release required for a therapeutically active agent, such as an opioid, and whether or not at least one polyalkylene glycol is present. In the absence of at least one polyalkylene glycol, the amount of the at least one aliphatic alcohol can range from about 20% to about 50% (by wt). When at least one polyalkylene glycol is present, the combined weight of the at least one aliphatic alcohol and the at least one polyalkylene glycol can range from about 20% to about 50% (by wt) of the total weight of the core.

Another example of a suitable controlled release matrix comprises an alkylcellulose, such as ethyl cellulose, a C₁₂ to C₃₆ aliphatic alcohol, and optionally a polyalkylene glycol.

In order to facilitate the preparation of a controlled release oral dosage form according to the invention, any method of preparing a matrix formulation known in the formulation art may be used. In one aspect of the invention, the controlled release core is in the form of a tablet composed of particles comprising at least one opioid agonist, and optionally at least one opioid antagonist, dispersed within a controlled release matrix. For example, incorporation of at least one opioid agonist, and optionally at least one opioid antagonist, in a controlled release matrix is accomplished by (a) forming granules comprising at least one water-soluble hydroxyalkyl cellulose and at least one opioid agonist, and optionally at least one opioid antagonist, (b) mixing the hydroxyalkyl cellulose containing granules with at least one C₁₂-C₃₆ aliphatic alcohol, and (c) optionally, compressing and shaping the granules. The granules can be formed by wet granulating the hydroxyalkylcellulose/opioid agonist or hydroxyalkylcellulose/opioid agonist/opioid antagonist with water. In this process, the amount of water added during the wet granulation step can be between 1.5 and 5 times, such as between 1.75 and 3.5 times, the dry weight of the opioid.

Controlled release matrices can also be prepared via melt-granulation or melt-extrusion techniques. Generally, melt-granulation techniques involve melting a normally solid hydrophobic material, e.g. a wax, and incorporating a powdered drug therein. To obtain a controlled release dosage form, it may be necessary to incorporate an additional hydrophobic substance, e.g. ethylcellulose or a water-insoluble acrylic polymer, into the molten wax hydrophobic material. Examples of controlled release formulations prepared via melt-granulation techniques are found in U.S. Pat. No. 4,861,598, the disclosure of which is hereby incorporated by reference.

An example of a method of preparing a suitable melt-extruded matrix is described, for example, in U.S. Pat. No. 6,288,398, the disclosure of which is incorporated herein by reference. The method is multi-step which first involves blending a therapeutically active agent, such as an opioid agonist, and optionally an opioid antagonist, together with at least one hydrophobic controlled release material to obtain a homogeneous mixture. The homogeneous mixture is then heated to a temperature sufficient to at least soften the mixture sufficiently to extrude the same. The resulting homogeneous mixture is then extruded to form strands. The cooled, hardened strand may be comminuted to produce a multiparticulate intermediate with the desired pellet size, shape and size distribution. Common types of comminutors that may be employed include cutters, choppers, grinders, mills, etc. The resulting pellets additionally may be shaped into spheres by a spheronization process. Changing the diameter of the die modifies the aspect ratio of the pellets or diameter of the resulting spheres. The extrudate preferably has a diameter of from about 0.1 to about 5 mm. Multiparticulates comprising the therapeutically active agents and the hydrophobic controlled release material provide sustained release for a time period of from about 8 to about 24 hours.

The controlled release core of dosage forms according to the invention can be in the form of liquids, tablets, or capsules comprising at least one multiparticulate which comprises at least one controlled release material and at least one therapeutically active agent.

Dosage forms comprising multiparticulate formulations have been described, for example, in U.S. Pat. No. 6,066,339 and U.S. Pat. No. 5,681,584, the disclosures of which are incorporated herein by reference. U.S. Pat. No. 6,066,339 describes an oral multiparticulate formulation comprising sustained release particle, wherein each particle has a core containing water soluble morphine and an osmotic agent, and wherein the core is coated with a rate-controlled polymer comprised of ammonia methacrylate polymers. U.S. Pat. No. 5,681,584 describes a delay jacket coating over a core, which comprises a therapeutically active agent, with an osmotic agent. Osmotic agents refer to a pharmaceutically acceptable material that enhances the passage of the water soluble therapeutically active agent, such as morphine, through the rate-controlling polymer coat or through the tissue in the gastrointestinal tract. Osmotic agents may act to enhance the absorption of a water soluble therapeutically active agent, such as morphine, by creating a local pH and/or chemical potential environment. Osmotic agents can comprise of at least one of the following: an organic acid, a pharmaceutically acceptable salt, or a gastrointestinal absorption enhancer. Suitable osmotic agents include, but are not limited to, adipic acid, ascorbic acid, citric acid, fumaric acid, malic acid, succinic acid, tartaric acid, lactic acid, monopotassium citrate, potassium acid tartrate, sodium fumarate, sodium dihydrogen phosphate, sodium bisulfate, sodium metabisulfate, or combinations thereof. Rate-controlled polymers compatible for use in the multiparticulate formulation disclosed in U.S. Pat. No. 6,066,339 include, for example, ammonia methacrylate copolymer type A and ammonia methacrylate copolymer type B as described in USP/NF in a ratio of from about 15:85 to about 1:99, respectively, such as a ratio of 5:95. Additional rate-controlled polymers compatible for use in the multiparticulate formulation disclosed in U.S. Pat. No. 6,066,339 include, for example, Eudragit RL and Eudragit RS in a ratio of about 5:95, respectively, such as a ratio of 12.5:12.5.

The controlled release core of dosage forms according to the invention can be in the form of liquids, tablets, or capsules comprising at least one multiparticulate which comprises (i) at least one controlled release material, (ii) at least one therapeutically active agent, and (iii) at least one osmotic agent.

Another example of a method of preparing a suitable melt-extruded matrix includes the steps of (i) directly metering into an extruder a homogeneous mixture comprising at least one hydrophobic controlled release material, at least one therapeutically active agent such as an opioid agonist, and optionally at least one therapeutically active agent, such as at least one opioid antagonist, and optionally at least one binder, (ii) heating the homogenous mixture, (iii) extruding the homogenous mixture to form strands, (iv) cooling the strands, and (v) cutting the strands into particles having a size from about 0.1 mm to about 12 mm. A relatively continuous manufacturing procedure is described, for example, in WO 01/58447 A1, the disclosure of which is incorporated herein by reference. The diameter of the extruder aperture or exit port can be adjusted to vary the thickness of the extruded strands and the exit part of the extruder can be any shape, such as round, oblong, or rectangular, for example. The exiting strands can be reduced to particles using a hot wire cutter, guillotine, etc.

The melt extruded multiparticulate system can be, for example, in the form of granules, spheroids or pellets depending upon the extruder exit orifice. Melt-extruded multiparticulate(s) and melt-extruded multiparticulate system(s) and melt-extruded particles can refer to a plurality of units, including within a range of similar size and/or shape and containing one or more active agents and one or more excipients, and/or including a hydrophobic material as described herein. In this regard, the melt-extruded multiparticulates can be of a range of from about 0.1 to about 12 mm in length and have diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

The controlled release core can be prepared in an oral dosage form of an effective amount of melt-extruded multiparticulates within a capsule. For example, a plurality of the melt-extruded multiparticulates may be placed in a gelatin capsule in an amount sufficient to provide an effective controlled release dose when ingested and contacted by gastric fluid.

The controlled release core can be prepared in an oral dosage form of an effective amount of melt-extruded multiparticulates that are compressed into an oral tablet using conventional tableting equipment using standard techniques. Techniques and compositions for making tablets (compressed and molded), capsules (hard and soft gelatin) and pills are also described in Remington's Pharmaceutical Sciences, (Arthur Osol, editor), 1553-1593 (1980), incorporated by reference herein.

The controlled release core can be prepared in an oral dosage form of an effective amount of melt-extruded multiparticulates that are compressed into an oral tablet as set forth in U.S. Pat. No. 4,957,681, the disclosure of which is hereby incorporated by reference.

The controlled release melt-extruded multiparticulates can be further coated with at least one hydrophobic controlled release material. The amount of the hydrophobic controlled release material in the additional coating can be sufficient to obtain a weight gain ranging from about 2% to about 30% (by weight), although the exact amount in the additional coat may be greater depending upon the physical properties of the particular therapeutically active agent utilized in the controlled release core and the desired release rate of the therapeutically active agent, for instance.

The controlled release core is in the dosage form of a capsule comprising a first melt-extruded multiparticulate and a second melt-extruded multiparticulate which comprises at least one therapeutically active agent different from the therapeutically active agent of the first melt-extruded multiparticulate. The controlled release core can be in a dosage form comprising an amount of an immediate release therapeutically active agent, including, for example, an opioid agonist, and optionally opioid antagonist, for prompt therapeutic effect. The controlled release core can be in a dosage form comprising a combination of beads comprising controlled release materials and matrix multiparticulates.

The release profile of the melt-extruded formulations can be altered, for example, by varying the amount of retardant, i.e., hydrophobic material, by varying the amount of plasticizer relative to hydrophobic material, by the inclusion of additional ingredients or excipients, by altering the method of manufacture, etc.

The melt-extruded material can be prepared without the inclusion of particles comprising a therapeutically active agent, which is added thereafter to the extrudate. Such formulations typically will have the therapeutically active agent blended together with the extruded matrix material, and then the mixture could be tableted in order to provide a slow release of the therapeutically active agent. Such formulations may be advantageous, for example, when the therapeutically active agent included in the formulation is sensitive to temperatures needed for softening the controlled release material and/or the retardant material.

The core can be in the form of granulates or particulates comprising different therapeutically active agents including, for example, an opioid agonist dispersed in a first controlled release matrix and an opioid antagonist dispersed in a second controlled-release matrix, wherein the controlled release matrix may be the same or different, and wherein the first and second matrices release different therapeutically active agents including, for example, the opioid agonist and the opioid antagonist, respectively, at substantially the same rate. The core can be in the form of granulates comprising different therapeutically active agents including, for example, an opioid agonist, and optionally an opioid antagonist, dispersed in a controlled-release matrix and further comprising an additional controlled release material.

Where the core comprises at least one therapeutically active agent that is coated with at least one controlled release material, the controlled release material coating can be chosen so as to achieve, in combination with the other stated properties, desired in vitro dissolution rates of the therapeutically active agent, including within the preferred ranges disclosed herein. The controlled release coating should be capable of producing a strong, continuous film that is smooth and elegant, capable of supporting pigments and other coating additives, non-toxic, inert, and tack-free. The controlled release coating can be at least one hydrophobic material selected from (i) an alkylcellulose; (ii) an acrylic polymer; or (iii) mixtures thereof. The coating can be applied in the form of an organic or aqueous solution or dispersion. The coating can be applied to obtain a weight gain from about 2% to about 25% of the controlled release dosage form in order to obtain a desired sustained release profile. Coatings derived from aqueous dispersions are described, for example, in U.S. Pat. Nos. 5,273,760 and 5,286,493, the disclosure of which are hereby incorporated by reference. Other examples of controlled release formulations and coatings which may be used in accordance with the present invention include U.S. Pat. Nos. 5,324,351, 5,356,467, and 5,472,712, the disclosures of all of which are incorporated by reference in their entirety.

Many methods, such as spray coating, for example, can be employed to coat the core with controlled release materials. In one possible method, a Wurster fluidized-bed system is used in which an air jet, injected from underneath, fluidizes the core material and effects drying while the controlled release material coating, such as acrylic polymer, is sprayed on with a sufficient amount of the controlled release material, so as to obtain a predetermined controlled release of the therapeutically active agent when the coated core is exposed to aqueous solutions, for example, a gastric millieux, such as gastric fluid. In determining the sufficient amount of coating, factors such as the physical characteristics of the therapeutically active agent, the manner of incorporation of the plasticizer, etc. are taken into account. After coating with the controlled release material, an additional overcoat of a film-former, such as Opadry® can be optionally applied to the beads. This overcoat can be provided, if at all, to substantially reduce agglomeration of the beads.

The controlled release core can be in the form of spheroids or beads for encapsulation. Such beads comprise at least one therapeutically active agent, including for example, at least one opioid agonist and optionally, at least one opioid antagonist, which are then subsequently coated with a hydrophobic controlled release material. Such hydrophobic materials include, for example, cellulosic materials and polymers, such as alkylcelluloses. A plurality of such resultant spheroids or beads can thereafter be placed in a gelatin capsule optionally with at least one therapeutically active agent, such as an opioid antagonist in a substantially non-releasable form. This dosage form provides an effective controlled release dose of the therapeutically active agent, such as an opioid agonist, when ingested and contacted by an environmental fluid, e.g., gastric fluid or dissolution media.

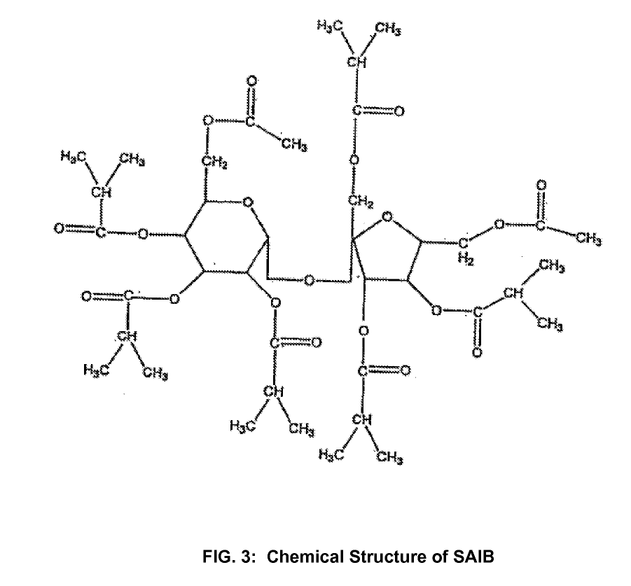

Preferred controlled release materials useful according to the invention include non-polymeric, non-water soluble high-viscosity liquid carrier materials (HVLCM) of viscosity of at least 5,000 cP at 37° C. which do not crystallize neat under ambient or physiological conditions. HVLCMs are described, for example, in U.S. Pat. No. 5,747,058, the disclosure of which is incorporated by reference herein. This HVLCM release material and at least one therapeutically active agent comprise a controlled released core according to a preferred aspect of the invention. A particularly preferred HVLCM is sucrose acetate isobutyrate (SAIB). SAIB is a modified sucrose molecule containing two acetic acid and six isobutyric moieties. The structure of SAM is shown as FIG. 3. Thus, in a preferred aspect, the controlled release core comprises a controlled release material that is an HVLCM according to U.S. Pat. No. 5,747,058 and a substance to be delivered, wherein the HVLCM is SAIB. In other embodiments, the controlled release core comprises a HVLCM that is a stearate ester such as those of propylene glycol, glyceryl, diethylaminoethyl, glycol, stearate amides and other long-chain fatty acid amide, such as N,N′-ethylene distearamide, stearamide MEA and DEA, ethylene bistearamide, cocoamine oxide, long chain tfatty alcohols, such as cetyl alcohol and stearyl alcohol, long-chain esters such as myristyl myristate, beheny erucate, and glyceryl phosphate. In an embodiment, the HVLCM is acetylated sucrose distearate (Crodesta A-10).

SAIB is orally non-toxic and has been used to stabilize emulsions in the food industry. It is a very viscous liquid and has an unusual property that there is a dramatic change in viscosity with small additions of heat or with the addition of solvents. It is soluble in a large number of biocompatible solvents. When in solution or in an emulsion, SAIB can be applied via injection or an aerosol spray. SATE is compatible with cellulose esters and other polymers that can affect the rate of delivery of the substance.

Biocompatible solvents to be used with an HVLCM such as SAIB include ethanol, dimethysulfoxide, ethyl lactate, ethyl acetate, benzyl alcohol, triacetin, N-methylpyrrolidone, propylene carbonate, glycofurol, freons such as trichlorofuloromethan and dichloromethane, dimethyl ether, propane, butane, dimethyl formamide, dimethyl acetamide, diethylene carbonate, butylenes glycol, N-(beta-hydromethyl)lactamide, dioxolanes, and other amides, esters, ethers, alcohols, to form a lower viscosity liquid carrier material (LVLCM) which is mixed with the substance (e.g. therapeutically active agent) to be delivered. In an embodiment, the LVLCM has a viscosity less than 1000 cP. On administration the controlled release core comprising the LVLCM is encapsulated with an immediate release gelatin capsule and is placed into the body, and the solvent dissipates or diffuses away from the LVLCM, forming in-situ a highly viscous composition that release the substance over time. By appropriate selection of the solvent and the HVLCM, a wide variety of pre- and post-administration composition viscosities can be achieved. In a preferred aspect, the HVLCM is biodegradable. Biocompatible solvents can be added to SAIB to obtain a resultant product with a desired viscosity. Biocompatible solvents can be added in an amount ranging from about 5% to about 55% by weight, relative to the total weight of the composition, such as from about 10% to about 50%, further such as from about 10% to about 30%. The amount of SAIB in the controlled release core of the invention is determined by the effect desired. The amount of SAIB in the controlled release core can range from 99.5% to 0.01% by weight (relative to the total weight of the controlled release core), such as from 99.5% to 10%, and such as from 95% to 25%, and such as from 85% to 45%, and further such as from 10% to 0.01%, and further such as from 2% to about 0.1%.

The controlled release core comprises SAIB and at least one biocompatible solvent, preferably ethanol, and at least one therapeutically agent. The amount of SAIB and the biocompatible solvent, preferably ethanol, is optimized by routine experimentation to achieve a particular desired viscosity. For example, a low viscosity solution that can be expelled from a glass pipet is obtained with a mixture containing 9 g of SAIB combined with 1 g of ethanol whereas, a thin film that can retain its shape for more than one week is obtained with a mixture containing 8 g of SAIB combined with 1 g of ethanol. The amount and type of the solvent used with SAM display varying viscosities, as can be measured using a Cannon-Fenske viscometer of size 200 at 30° C. For example, compositions comprising SAIB with ethanol in a ratio of 60:40, 70:30, and 90:10 exhibit centipoises values of 7,7,17.0, and 494.8, respectively. In comparison, ethanol only exhibits a centipoise value of 1.3. Also, compositions comprising SAIB, ethanol, and cellulose acetate butyrate (CAB) in a ratio of 55:40:5 respectively exhibits a centipoise value of 68.9.

Where the controlled release core is in the form of a liquid, the amount and type of solvent used with SAIB should be optimized so as to obtain a liquid wherein the at least one therapeutically active agent is acceptably soluble. For example, formulations comprising small organic molecules, such as ibuprofen, require approximately 15% (by weight) of ethanol in order to achieve solubility with SAIB whereas, formulations comprising large peptidic molecules such as bovine serum albumin, do not solubilize with about 40% ethanol, even with the addition of co-solvents, such as glycerol and/or DMSO. Another example is a composition comprising SAIB and naproxen (sodium salt), which requires glycolfurol as a solvent so as to achieve solubility because naproxen is not soluble in ethanol and ethylacetate.

In a preferred aspect, the amounts of (i) SAIB, (ii) at least one biocompatible solvent and (iii) oxycodone alone or optionally, with naltrexone are optimized to achieve a desired viscosity. Preferred solvents for SAIB formulations with small organic molecules include, but are not limited to, ethanol, glycofurol, ethylacetate, ethylacetate, N-methyl-pyrrolidone, and propylene carbonate. Optionally, cosolvents, such as dimethylsulfoxide, or glycers may be added to enhance the solubility. However, the amount and type of solvent(s) with SAIB formulations are optimized with the oxycodone alone and optionally, naltrexone that is to be formulated. In this example, the amount of SAIB and the amount and type of biocompatible solvent(s) used with oxycodone alone or optionally, with naltrexone is optimized to produce a resultant liquid mixture of (i) SAIB, (ii) biocompatible solvent(s), and (iii) oxycodone alone or, optionally, with naltrexone, is pharmaceutically acceptable for encapsulation and/or tabulation.

A variety of additives can optionally be added to the HVLCM or LVLCM to modify the properties of the therapeutically active agent as desired. The additives can be present in any amount which is sufficient to impart the desired properties to the composition. The amount of additive used will in general be a function of the nature of the additive and the effect to be achieved, and can be readily determined by routine experimentation. Non-limiting examples of additive include, for instance, biodegradable polymers and oligomers that can be used to alter the release profile of the therapeutically active agent, non-biodegradable polymers, natural and synthetic oils and fats, and carbohydrate and carbohydrate derivatives. For example, at least one additive may be included in the oxycodone/SAIB or oxycodone/naltrexone/SAIB. Such additives include, for example, cellulose acetate butyrate (CAB), cellulose acetate propionate (CAP), PVP, PVP-25, PEG-10K, PEG-1K, and sucrose. Again, the amount and type of additive(s) should be optimized. In a preferred aspect, the amount and type of additive(s) used with oxycodone alone or optionally, with naltrexone is optimized to produce a resultant liquid mixture of (i) SAIB, (ii) at least one biocompatible solvent, and (iii) oxycodone alone or, optionally, with naltrexone, and (iv) additive, wherein the mixture is pharmaceutically acceptable for encapsulation and/or tabulation.

The controlled release core can comprise SAIB that is loaded into an aerosol container and sprayed onto agar plates to form an adhesive continuous film. In another aspect, the controlled release core comprising SAIB is sprayed onto gelatin. In yet another aspect, the controlled release core comprising SAIB is loaded into a syringe equipped with a gauged needle and extruded.

The controlled release core of the invention comprises at least one of any therapeutically active agent. The at least one therapeutically active agent in the controlled release core can be the same or different from the at least one therapeutically active agent in the immediate release gelatin capsule coating. In an aspect of the invention, the therapeutically active agent of the controlled release comprises at least one opioid agonist. In a preferred aspect of the invention, the therapeutically active agent of the controlled release is oxycodone. In another aspect of the invention, the therapeutically active agent of the controlled release is a combination of at least one opioid agonist and at least one opioid antagonist. In another preferred aspect of the invention, the therapeutically active agent of the controlled release is a combination of oxycodone and naltrexone.

When the core comprises a hydrophobic therapeutically active agent, the controlled release core can comprise a carrier system to aid in formulation. Such carrier systems are described, for example, in U.S. Pat. No. 6,096,338, the disclosure of which is incorporated herein by reference. The carrier system can comprise, for example, at least one digestible oil and at least one pharmaceutical acceptable surfactant, wherein the surfactant comprises at least one hydrophilic component that substantially inhibits in vivo lipolysis of the digestible oil, and wherein the surfactant comprises at least one lipophilic component that substantially reduces the inhibitory effect of the hydrophilic surfactant component. Non-limiting examples of surfactants include fatty acids, such as oleic acid, mono- and/or di-glycerides of fatty acids, such as capric/caprylic acid, acetic, succinic, lactic, citric, and/or tartaric esters, propylene glycol, castor oil ethoxylates, and sorbitan esters of fatty acids.

The amount of the therapeutically active agent in association with a controlled release material in the core is sufficient to facilitate a sustained, desired biological effect for a prolonged period of time, such as from about 2 hours to about 24 hours, and such as from about 8 hours to about 24 hours. The amount of the therapeutically active agent in the controlled release core can also depend upon the desired release profile and the concentration of drug required for a desired biological effect. Additional factors used to determine the amount of the therapeutically active agent in the controlled release core include absorption, inactivation, and excretion rates of the therapeutically active agent, as well as other factors known to those of ordinary skill in the art.

The controlled release core is formulated in a pharmaceutically acceptable oral dosage form, such as a liquid, capsule, or tablet. Exact dimensions and size of the controlled release core of the present invention can be optimized within the scope of routine experimentation.

The therapeutically active agent in the controlled release core is an opioid agonist alone, such as oxycodone, present in an analgesic or subanalgesic (e.g. non-analgesic) amount in a human subject. The agonist may also be present in an amount that is anti-analgesic in the human subject. In a preferred aspect, the amount of the opioid agonist, alone, in the controlled release core is from about 0.1 to about 300 mg.

The therapeutically active agent in the controlled release core is a combination of an opioid antagonist and an opioid agonist, which is present in a subanalgesic amount. In a preferred aspect, the controlled-release oral dosage form provides a controlled release of an opioid agonist and a controlled-release of an opioid antagonist, such that when the dosage form is administered to a human, the blood levels of the agonist is maintained throughout the dosing period at an analgesically effective level, and the antagonist at a level sufficient to decrease the side effects associated with the opioid agonist but not sufficient to negate the analgesic effect of the opioid agonist.

The therapeutically active agent of the controlled release core is a combination of oxycodone and naltrexone, wherein oxycodone is present in an amount of about 0.1 to about 300 mg, and wherein the naltrexone is provided in an amount of about 0.000001 to about 1.0 mg, alternatively less than about 1.0 mg, alternatively less than about 0.5 mg. When the opioid antagonist is used in combination with the opioid agonist, the amount of the opioid agonist administered can be an analgesic or sub-analgesic amount (e.g., non-analgesic) in the human subject. Alternatively, the opioid agonist can be present in an amount that is anti-analgesic in the human subject. The opioid antagonist in the controlled release core is present in an amount of about 0.000001 to about 1.0 mg, alternatively less than about 1.0 mg, alternatively less than about 0.5 mg. Preferred ranges of opioid antagonists also include: from about 0.000001 mg to less than 1.0 mg; from about 0.00001 mg to less than 1.0 mg, from about 0.0001 mg to less than 1.0 mg; from about 0.001 mg to less than 1.0 mg; from about 0.01 mg to less than 1.0 mg; or from about 0.1 mg to less than 1.0 mg. Additional preferred ranges of opioid antagonists include: from about 0.000001 mg to less than 1.0 mg; from about 0.00001 mg to less than 1.0 mg, from about 0.0001 mg to about 0.1 mg; from about 0.001 mg to about 0.1 mg; from about 0.01 mg. to about 0.1 mg; from about 0.001 mg to about 0.1 mg; from about 0.001 mg to about 0.01 mg; or from about 0.01 mg to about 0.1 mg. Further preferred ranges of opioid antagonists include: from about 0.000001 mg to less than 1.0 mg; from about 0.00001 mg to less than 1.0 mg, from at least about 0.0001 to less than about 0.5 mg; from at least about 0.01 to less than about 0.5 mg; or from at least about 0.1 to less than about 0.5 mg. Alternatively, the maximum amount of opioid antagonist in the immediate release gelatin capsule is 1 mg. Alternatively, the maximum amount of opioid antagonist in the immediate release gelatin capsule is less than 0.5 mg. The minimum amount of opioid antagonist in the immediate release gelatin capsule is 0.000001 mg. Any minimum amount and any maximum amount of antagonist in the immediate release gelatin capsule, as specified above, may be combined to define a range of amounts in increments of 0.000001 or 0.00001 or 0.0001 or 0.001 or 0.01 or 0.1, providing that the minimum selected is equal to or less than the maximum selected. In an embodiment of the invention, the amount of antagonist in the immediate release gelatin capsule is less than an effective amount to antagonize an exogenous or endogenous opioid agonist, but such an amount may include an amount that enhances the potency and/or attenuates an adverse effect of the agonist, including, for example, nausea, vomiting, headache, dizziness, somnolence, pruritus, tolerance, withdrawal, dependence, and/or addiction.

In a preferred aspect of the invention, the controlled release core is optionally further coated with an enteric coating that is affixed between the controlled release core and the immediate release gelatin capsule coating. In this embodiment, the therapeutically active agent in the immediate release gelatin capsule coating is immediately released into the gastric juices of the stomach and the enteric coating protects the controlled release core allowing passage of the controlled release core through the stomach and into the basic environment of the duodenum. Upon dissolution of the enteric coat in the duodenum, the therapeutically active agent of the controlled release core is released. This embodiment provides a significantly stable drug concentration profile in the plasma, and is especially beneficial for therapeutically active agents that have narrow therapeutic windows and require multiple daily closings. Moreover, in this embodiment, the therapeutically active agent of the controlled release core exhibits an absorption profile wherein the period of time in which MEC levels are maintained ranges from at least about eight hours, such as from at least about twelve hours, to up to about twenty-four hours in a human subject.

Enteric coating includes any coating or layering which serves to resist disintegration in the stomach and permits the component to pass intact into the duodenum or to be delayed in release. Enteric gelatin capsules with at least one therapeutically active agent are useful in dosage forms of the invention. Such encapsulation materials and methods without an active agent are available from Banner Pharmacaps as their Gelatin Binary System™.

Enteric layers or coatings are described, for example, in U.S. Pat. No. 5,968,554, the disclosure of which is incorporated herein by reference. Enteric layers or coatings do not dissolve in the acidic environment of the stomach, but do dissolve at a pH of 5.0 or higher. A variety of materials can be used for such enteric layers or coatings, as long as they do not readily dissolve or disperse in the gastric juices of the stomach and do dissolve or disperse in the intestinal fluid.

Non-limiting examples of materials that can be used for enteric layers or coating include, for example, polymeric acids and mixtures of polymeric acids, shellac, cetyl alcohol, and cellulose acetate. Other representative enteric layers or coating include, for example, polymers of ethylcellulose, hydroxypropycellulose, and carbomethylcellulose. Additional representative enteric layers or coating include, for example, shellac, cellulose acetate phthalate (CAP), polyvinyl acetate phthalate (PVAP), hydroxypropylmethylcellulose phthalate, and methacrylic acid ester copolymers, zein, and the like. Blends of various enteric polymers can also be used. Other non-limiting examples of materials useful in enteric layers or coatings include, for example, acrylic resins, wax, or other film forming materials that will dissolve or disperse in the intestine but remain intact in the stomach. An enteric polymer coating may be applied with or without solvent to the substrate that contains the agent, for example a drug. The pharmaceutic process may include spray coating, spray drying and press coating. An enteric coating comprising a water-based emulsion polymers can also be used. An enteric coating that can be used in the present invention can be ethylacrylate methacrylic acid copolymers sold under the trademark Eudragit® by Rhom GmbH of Domstadt, Germany. One type of enteric coating is Eudragit®L30D, which has a molecular weight of about 250,000 and is generally applied as a 25-75% aqueous solution. Another type of enteric coating is Eudragit® L30D-55 and is applied as a 45-55% weight aqueous solution. Other types of Eudragits® that may be used for enteric layers or coating include HP50, HP55, L100, and S100.

Certain methacrylic acid ester-type polymers are useful for preparing enteric coating. For example, there are a family of copolymers synthesized from diethylaminoethyl methacrylate and other neutral methacrylic esters, also known as methacrylic acid copolymer or polymeric methacrylates, commercially available as Eudragit® from Rohm Tech, Inc. There are several different types of Eudragit® that are suitable for use as enteric coatings. For example, Eudragit® E is an example of a methacrylic acid copolymer which swells and dissolves in acidic media. Eudragit® L is a methacrylic acid copolymer which does not swell at about pH<5.7 and is soluble at about pH>6. Eudragit® S does not swell at about pH<6.5 and is soluble at about Eudragit® RL and Eudragit® RS are water swellable, and the amount of water absorbed by these polymers is pH-dependent, however, dosage forms coated with Eudragit® RL and RS are pH-independent.

An oral dosage form according to the invention of the controlled release core and/or immediate release gelatin capsule may further include, in addition to a therapeutically active agent, including, for example, an opioid agonist and optionally an opioid antagonist, one or more drugs that may or may not act synergistically with such agent(s). For example, a combination of two opioid agonists may be included in the dosage form, in addition to the opioid antagonist. For example, the dosage form may include two opioid agonists having different properties, such as half-life, solubility, potency, and a combination of any of the foregoing. Alternatively, one or more opioid agonists are included and a non-opioid drug is also included, alternatively or in addition to an opioid antagonist. However, non-opioid drugs can provide additional analgesia, and include, for example, aspirin, acetaminophen; non-steroidal anti-inflammatory drugs (“NSAIDS”), e.g., ibuprofen, ketoprofen, etc.; N-methyl-D-aspartate (NMDA) receptor antagonists, e.g., a morphinan such as dextromethorphan or dextrorphan, or ketamine; cycboxygenase-II inhibitors (“COX II inhibitors”); cyclooxygenase-III inhibitors (“COX-III inhibitors”) and/or glycine receptor antagonists.

For example, lower doses of the opioid analgesic can be used by virtue of the inclusion of an additional non-opioid agonist, such as an NSAID or a COX-2 inhibitor. By using lower amounts of either or both drugs, the side effects associated with effective pain management in humans can be reduced.

Suitable non-steroidal anti-inflammatory agents, including ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like. Useful dosages of these drugs are well known in the art.

N-methyl-D-aspartate (NMDA) receptor antagonists are well known in the art, and encompass, for example, morphinans such as dextromethorphan or dextrorphan, ketamine, d-methadone or pharmaceutically acceptable salts thereof. NMDA antagonist encompasses drugs that block a major intracellular consequence of NMDA-receptor activation, e.g. a ganglioside such as GM.sub.1 or GT.sub.1b a phenothiazine such as trifluoperazine or a naphthalenesulfonamide such as N-(6-aminothexyl)-5-chloro-1-naphthalene-sulfonamide. These drugs are stated to inhibit the development of tolerance to and/or dependence on addictive drugs, e.g., narcotic analgesics such as morphine, codeine, etc. in U.S. Pat. Nos. 5,321,012 and 5,556,838 (both to Mayer, et al.), and to treat chronic pain in U.S. Pat. No. 5,502,058 (Mayer, et al.), all of which are hereby incorporated by reference. In addition, antagonist(s), of other glutomate receptor subtypes, e.g., AMPA, kainite or metabotropic glutamate receptors, or of glutamate receptor subunits for the treatment of pain, tolerance or action. The NMDA or other glutomate receptor subtypes antagonist may be included alone, or in combination with a local anesthetic such as lidocaine, as described in these Mayer, et al. patents. Analgesic immediate and controlled release pharmaceutical compositions of NMDA receptor antagonists and methods for treating pain with such compositions are described in U.S. Pat. No. 6,194,000, which is hereby incorporated by reference.

The NMDA receptor antagonist may be selected from a morphinan such as destromethorphan and dextrorphan, ketamine, amantadine, memantine, eliprodil, ifenprodil, dizocilpine, remacemide, iamotrigine, riluzole, aptiganel, phencyclidine, flupirtine, celfotel, felbamate, spermine, spermidine, levemopamil, a pharmaceutically acceptable salt or ester thereof, or a metabolic precursor of any of the foregoing.

The formulation may include sufficient NMDA receptor antagonist to provide from about 1-5000 mg/day, typically 1-1000 mg/day and preferably about 100-800 mg/day of the active ingredient. The composition includes an NMDA receptor antagonist in an immediate release form in association with a NMDA receptor antagonist in a controlled release form. The composition may include an amount of NMDA receptor antagonist in the immediate release form of approximately 5% to 90% of the total NMDA receptor antagonist, preferably 10% to 60%. An immediate release NMDA receptor antagonist content of about 15% to 50% is particularly preferred. The controlled release form of the NMDA receptor antagonist may constitute the remainder of the active ingredients.

The treatment of chronic pain via the use of glycine receptor antagonists and the identification of such drugs is described in U.S. Pat. No. 5,514,680 (Weber, et al.), hereby incorporated by reference.

COX-2 inhibitors have been reported in the art and many chemical structures are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944; and 5,130,311, all of which are hereby incorporated by reference. Certain preferred COX-2 inhibitors include valdecoxib (also known as Bextra), celecoxib (SC-58635, also known as Celebrex), DUP-697, flosulide (CGP-28238), meloxicam, 6-methoxy-2 naphthylacetic acid (6-MNA), MK-966 (also known as Vioxx), nabumetone (prodrug for 6-MNA), nimesulide, NS-398, SC-5766, SC-58215, T-614; or combinations thereof. Dosage levels of COX-2 inhibitor on the order of from about 0.005 mg to about 140 mg per kilogram of body weight per day can be therapeutically effective in combination with an opioid analgesic. Alternatively, about 0.25 mg to about 7 g per patient per day of a COX-2 inhibitor can be administered in combination with an opioid analgesic. COX-3 inhibitors have also been reported in the art and are useful in dosage forms according to the invention (Chandrasekhara, et al., 2002, Proc. Medl. Acad. Sci. USA 99: 13926-31).

Additionally or alternatively, a non-opioid drug can be included which provides a desired effect other than analgesia, e.g., antiitussive, expectorant, decongestant, antihistamine drugs, local anesthetics, and the like. Improved controlled release oral dosage forms according to the invention comprise an opioid agonist and an opioid antagonist in combination with a non-opiod drug, for example, acetominophen. Acetaminophen is an analgesic/antipyretic drug that has been utilized for treating mild to moderate pain such as headache, neuralgia, and musculoskeletal pain. The recommended daily adult dose is about 325 to about 650 mg every 4 hours, not to exceed a total dose of 4 g in 24 hours. The maximum dose of immediate release acetaminophen is generally considered to be about 1000 mg. Combination formulations can include such acetaminophen doses as those set forth above, or lower doses per 4 hour dosing interval. Thus, it is possible that controlled release formulations prepared in accordance with the present invention include a greater total acetominophen dose than the 325-650 mg dose, but that dose will be released in a controlled-release manner over a longer dosing interval (e.g., over 8 hours or more).

It is contemplated that the dosage of acetaminophen and opioid agonist in the formulations and method of the present invention may be similar or the same as dosages which are already commercially available and accepted by clinicians. Acetaminophen is commercially available in the United States in fixed combination with opioid agonists, namely, codeine, oxycodone and hydrocodone. Typical oral capsule dosages of acetaminophen/codeine combinations include 325 mg acetaminophen and 15 mg codeine phosphate, 325 mg acetaminophen and 30 mg codeine phosphate and 325 mg acetaminophen and 60 mg codeine phosphate. Tablets typically include 300 mg acetaminophen and 7.5 mg codeine phosphate, 300 mg acetaminophen and 15 mg codeine phosphate, 300 mg acetaminophen and 30 mg codeine phosphate, and 300 mg acetaminophen and 60 mg codeine phosphate.

Hydrocodone/acetaminophen products are typically available in fixed combinations of 5 mg hydrocodone (as the bitartrate salt) and 500 mg acetaminophen. Hydrocodone/acetaminophen tablets are typically available in fixed combinations of 500 mg acetaminophen and 2.5 mg hydrocodone bitartrate, 500 mg acetaminophen and 5 mg hydrocodone bitartrate, 500 mg acetaminophen and 7.5 mg hydrocodone, 7.5 mg hydrocodone bitartrate and 650 or 750 mg acetaminophen, and 10 mg hydrocodone bitartrate and 500, 650, 660 mg acetaminophen. Oxycodone/acetaminophen capsules and caplets are available in fixed combination of 5 mg oxycodone (as the hydrochloride salt) and 500 mg acetaminophen, and in tablets as 5 mg oxycodone hydrochloride and 325 mg acetaminophen.

Fixed combination tablets may be useful as a source of therapeutically active agent(s) for formulation into dosage forms with controlled release cores that are enrobed with a gelatin capsule comprising therapeutically active agent(s).

The fixed combinations described above are for information purposes only and are not meant to limit the possible relative amounts of opioid and acetaminophen contained in the formulations encompassed within the present invention. As disclosed herein and in accordance with the present invention, it is contemplated that in certain embodiments, the opioid agonist/opioid antagonist/acetaminophen combinations encompassed herein will have greater or lesser dosages of either the opioid agonist or acetaminophen, and that the ratio of opioid agonist to acetaminophen will vary based on the particular opioid agonist and opioid antagonist chosen for a formulation and the amount of opioid antagonist included therein, among other things.

An oral dosage form can comprise an opioid agonist (hydrocodone or oxycodone) and opioid antagonist (naltrexone or nalmefene) and acetaminophen. A non-opioid drug also can be included which provides a desired effect other than analgesia, e.g., antitussive, expectorant, decongestant, antihistamine drugs, local anesthetics, and the like.

At least one therapeutically active agent in the immediate release gelatin capsule coating and/or at least one therapeutically active agent in the controlled release core of the present invention may be provided in the form of free bases or pharmaceutically acceptable acid addition salts. Pharmaceutically acceptable salts refer to derivatives of a therapeutically active agent, wherein the therapeutically active agent is modified by making an acid or base salts thereof. The pharmaceutically acceptable salt embraces an inorganic or an organic salt. Examples of pharmaceutically acceptable salts include, but are not limited to, mineral or organic acid salts of the therapeutically active agent. Non-limiting examples of pharmaceutically acceptable salts include, but are not limited to, metal salts such as sodium, potassium salt, secium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediame salt and the like; inorganic acid salts such as hydrochloride, hydrobromide, sulfate, phosphate and the like; organic acid salts such as formate, acetate, trifluoroacetate, maleatem tartarate and the like; sulfonates such as methanesulfonate, benzenesulfonate, p-toluenesulfonate, and the like; amino acid salts such as arginate, asparginate, glutamate and the like. The pharmaceutically acceptable salts include the conventional non-toxic salts made, for example, from nontoxic inorganic or organic acids. For example, such conventional non-toxic salts include those derived from inorganic acids such as hydrochloric, hydrobromic, sulfuric, sulfonic, sulfamic, phosphoric, nitric and others known to those skilled in the art; and the salts prepared from organic acids such as amino acids, acetic, propionic, succinic, glycolic, stearic, lactic, malic, malonic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, glucuronic, and other acids. Other pharmaceutically acceptable salts and variants include mucates, phosphate (dibasic), phosphate (monobasic), acetate trihydrate, bi(heptafluorobutyrate), bi(methylcarbamate), bi(pentafluoropropionate), mesylate, bi(pyridine-3-carboxylate), bi(trifluoroacetate), bitartrate, chlorhydrate, and sulfate pentahydrate. An oxide, though not usually referred to by chemists as a salt, is also a “pharmaceutically acceptable salt” for the present purpose. For acidic compounds, the salt may include an amine-based (primary, secondary, tertiary or quaternary amine) counter ion, an alkali metal cation, or a metal cation. Lists of suitable salts are found in texts such as Remington's Pharmaceutical Sciences, 18^{th} Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, (Arthur H. Kibbe, ed.; Amer. Pharmaceutical Assoc., 2002); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: The Pharmacological Basis of Therapeutics 10th Ed. (Louis S. Goodman and Lee E. Limbird, eds.; McGraw Hill, 2002), the disclosures of which are hereby incorporated by reference.

Pharmaceutically acceptable refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The dosage form of the present invention including the immediate release gelatin capsule coating and/or the controlled release core may be compounded with at least one of the usual non-toxic, pharmaceutically acceptable excipients, carriers, diluents or other adjuvants. The choice of adjuvants will depend upon the active ingredients employed, the physical form of the composition, the route of administration, and other factors.

The excipients, binders, carriers, and diluents which can be used include water, glucose, lactose, natural sugars such as sucrose, glucose, or corn sweeteners, sorbitol, natural and synthetic gums such as gum acacia, tragacanth, sodium alginate, and gum arabic, gelatin, mannitol, starches such as starch paste, corn starch, or potato starch, magnesium trisilicate, talc, keratin, colloidal silica, urea, stearic acid, magnesium stearate, dibasic calcium phosphate, crystalline cellulose, methyl cellulose, carboxymethyl cellulose, polyethylene glycol, waxes, glycerin, and saline solution, among others.

Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The dosage form of the immediate release gelatin capsule coating and/or the controlled release core can also comprise at least one acidifying agent, adsorbent, alkalizing agent, antiadherent, antioxidant, binder, buffering agent, colorant, complexing agent, diluent, filler, direct compression excipient, disintegrant, flavorant, fragrance, glidant, lubricant, opaquant, plasticizer, polishing agent, preservative, sweetening agent, or other ingredients known for use in pharmaceutical preparations.

Acidifying agents include compounds used to provide an acidic medium for product stability. Such compounds include, by way of example and without limitation, acetic acid, amino acid, citric acid, fumaric acid and other alpha hydroxy acids, hydrochloric acid, ascorbic acid, nitric acid, phosphoric acid, and others known in the art.

Adsorbents include agents capable of holding other molecules onto their surface by physical or chemical (chemisorption) means. Such compounds include, by way of example and without limitation, powdered and activated charcoal, zeolites, and other materials known in the art.

Alkalizing agents include compounds used to provide an alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known in the art.

Antiadherents include agents that prevent the sticking of solid dosage formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known in the art.

Antioxidants include agents which inhibit oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate and sodium metabisulfite and other materials known in the art.

Binders include substances used to cause adhesion of powder particles in solid dosage formulations. Such compounds include, by way of example and without limitation, acacia, alginic acid, carboxymethylcellulose sodium, poly(vinylpyrrolidone), compressible sugar (e.g., NuTab), ethylcellulose, gelatin, liquid glucose, methylcellulose, povidone and pregelatinized starch and other materials known in the art.

When needed, binders may also be included in the dosage forms of the immediate release gelatin capsule coating and/or the controlled release core of the present invention. Exemplary binders include acacia, tragacanth, gelatin, starch, cellulose materials such as methyl cellulose, HPMC, HPC, HEC and sodium carboxy methyl cellulose, alginic acids and salts thereof, polyethylene glycol, guar gum, polysaccharide, bentonites, sugars, invert sugars, poloxamers (PLURONIC™ F68, PLURONIC™ F127), collagen, albumin, gelatin, cellulosics in nonaqueous solvents, combinations thereof and others known to those skilled in the art. Other binders include, for example, polypropylene glycol, polyoxyethylene-polypropylene copolymer, polyethylene ester, polyethylene sorbitan ester, polyethylene oxide, combinations thereof and other materials known in the art.

Buffering agents include compounds used to resist changes in pH upon dilution or addition of acid or alkali. Such compounds include, by way of example and without limitation, potassium metaphosphate, potassium phosphate, monobasic sodium acetate and sodium citrate anhydrous and dihydrate and other materials known in the art.

Sweetening agents include compounds used to impart sweetness to a preparation. Such compounds include, by way of example and without limitation, aspartame, (EQUAL®, sucralose (SPLENDA™), acesulfame K (Sunette® or Sweet One®), dextrose, glycerin, mannitol, saccharin sodium, sorbitol, sucrose, and other materials known in the art.

Diluents or fillers include inert substances used to create the desired bulk, flow properties, and compression characteristics in the preparation of solid dosage forms. Such compounds include, by way of example and without limitation, dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, and starch and other materials known in the art.

Direct compression excipients include compounds used in compressed solid dosage forms. Such compounds include, by way of example and without limitation, dibasic calcium phosphate (e.g., Ditab) and other materials known in the art.

Disintegrants include compounds used in solid dosage forms to promote the disruption of the solid mass into smaller particles that are more readily dispersed or dissolved. Exemplary disintegrants include, by way of example and without limitation, starches such as corn starch, potato starch, pre-gelatinized and modified starches thereof, sweeteners, clays such as bentonite, microcrystalline cellulose (e.g., Avicel), methyl cellulose, carboxymethylcellulose calcium, sodium carboxymethylcellulose, hydroxy propylcellulose-low substituted, colloidal silicon dioxide, alginic acid, sodium alginate, cellulose polyacrilin potassium (e.g., Amberlite), alginates, sodium starch glycolate, gums, agar, guar, locust bean, karaya, xanthan, pectin, tragacanth, agar, bentonite, polyvinylpyrrolidone and other materials known in the art.

Glidants are agents used in solid dosage formulations to promote flowability of the solid mass. Such compounds include, by way of example and without limitation, colloidal silica, cornstarch, talc, calcium silicate, magnesium silicate, colloidal silicon, tribasic calcium phosphate, silicon hydrogel and other materials known in the art.

Lubricants include substances used in solid dosage formulations to reduce friction during compression. Such compounds include, by way of example and without limitation, sodium oleate, sodium stearate, calcium stearate, zinc stearate, magnesium stearate, polyethylene glycol, talc, mineral oil, stearic acid, sodium benzoate, sodium acetate, sodium chloride, and other materials known in the art.

Opaquants include compounds used to render a coating opaque. An opaquant may be used alone or in combination with a colorant. Such compounds include, by way of example and without limitation, titanium dioxide, talc and other materials known in the art.

Polishing agents include compounds used to impart an attractive sheen to solid dosage forms. Such compounds include, by way of example and without limitation, carnauba wax, white wax and other materials known in the art.

Colorants include compounds used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, ferric oxide, other FD&C dyes and natural coloring agents such as grape skin extract, beet red powder, beta-carotene, annato, carmine, turmeric, paprika, and other materials known in the art. The amount of coloring agent used will vary as desired.

Flavorants include compounds used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those skilled in the art. Particularly contemplated flavors are the grape and cherry flavors and citrus flavors such as orange.

Complexing agents include, for example, EDTA disodium or its other salts and other agents known in the art.

Exemplary fragrances include those generally accepted as FD&C grade.

Exemplary preservatives include materials that inhibit bacterial growth, such as Nipagin, Nipasol, alcohol, antimicrobial agents, benzoic acid, sodium benzoate, benzyl alcohol, sorbic acid, parabens, isopropyl alcohol and others known in the art.

For example, where the controlled release core is in a solid dosage form, at least one surface active agents or cosolvents that improve wetting or disintegration of the core and/or layer and/or coating of the solid dosage form can be included.

The controlled release core and/or immediate release gelatin capsule coating can include plasticizers where plasticizers can be included to modify the physical, mechanical, and aesthetic properties of the polymers used in the coats or the dosage form. Plasticizers include compounds capable of plasticizing or softening a polymer or binder used. The plasticizer should be able to lower the melting temperature or glass transition temperature (softening point temperature) of the polymer or binder. Plasticizers, such as low molecular weight PEG, generally broaden the average molecular weight of a polymer in which they are included thereby lowering its glass transition temperature or softening point. Plasticizers also generally reduce the viscosity of a polymer. It is possible the plasticizer will impart some particularly advantageous physical properties to the dosage form of the invention.

Plasticizers useful in dosage forms according to the invention can include, by way of example and without limitation, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutylsebacate, acetyltributylcitrate, triethyl citrate, acetyl triethyl citrate, tributyl citrate and allyl glycolate. All such plasticizers are commercially available from sources such as Aldrich or Sigma Chemical Co. It is also contemplated and within the scope of the invention, that a combination of plasticizers may be used in the present formulation. The PEG based plasticizers are available commercially or can be made by a variety of methods, such as disclosed in Poly(ethylene glycol) Chemistry: Biotechnical and Biomedical Applications (J.M. Harris, Ed.; Plenum Press, NY) the disclosure of which is hereby incorporated by reference.

The controlled release core and/or immediate release gelatin capsule coating of the present invention can also include oils, for example, fixed oils, such as peanut oil, sesame oil, cottonseed oil, corn oil and olive oil; fatty acids, such as oleic acid, stearic acid and isostearic acid; and fatty acid esters, such as ethyl oleate, isopropyl myristate, fatty acid glycerides and acetylated fatty acid glycerides. It can also be mixed with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent. Soaps and synthetic detergents may be employed as surfactants and as vehicles for the dosage form. Suitable soaps include fatty acid alkali metal, ammonium, and triethanolamine salts. Suitable detergents include cationic detergents, for example, dimethyl dialkyl ammonium halides, alkyl pyridinium halides, and alkylamine acetates; anionic detergents, for example, alkyl, aryl and olefin sulfonates, alkyl, olefin, ether and monoglyceride sulfates, and sulfosuccinates; nonionic detergents, for example, fatty amine oxides, fatty acid alkanolamides, and poly(oxyethylene)-block-poly(oxypropylene) copolymers; and amphoteric detergents, for example, alkyl-aminopropionates and 2-alkylimidazoline quaternary ammonium salts; and others known in the art; and mixtures thereof.

A water soluble coat or layer can be formed to surround a solid dosage form or a portion thereof. The water soluble coat or layer can either be inert or drug-containing. Such a coat or layer will generally comprise an inert and non-toxic material which is at least partially, and optionally substantially completely, soluble or erodible in an environment of use. Selection of suitable materials will depend upon the desired behavior of the dosage form. A rapidly dissolving coat or layer will be soluble in the buccal cavity and/or upper GI tract, such as the stomach, duodenum, jejunum or upper small intestines. Exemplary materials are disclosed in U.S. Pat. No. 4,576,604 to Guittard et al. and U.S. Pat. No. 4,673,405 to Guittard et al., and U.S. Pat. No. 6,004,582 to Faour et al. and the text Pharmaceutical Dosage Forms: Tablets Volume I, 2^{nd} Edition. (A. Lieberman. ed. 1989, Marcel Dekker, Inc.), the disclosures of which are hereby incorporated by reference. In some embodiments, the rapidly dissolving coat or layer will be soluble in saliva, in the gastric millieux, gastric juices, or acidic fluids.

Materials which are suitable for making the water soluble coat or layer include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; croscarmellose sodium; other cellulose polymers such as sodium carboxymethylcellulose; and other materials known in the art. Other lamina-forming materials that can be used for this purpose include polyvinyl alcohol), poly(ethylene oxide), gelatin, glucose and saccharides. The water soluble coating can comprise other pharmaceutical excipients that may or may not alter the way in which the water soluble coating behaves. The above-noted materials include film-forming polymers.

A water soluble coat or layer can also comprise hydroxypropyl methylcellulose, which is supplied by Dow under its Methocel E-15 trademark. The materials can be prepared in solutions having different concentrations of polymer according to the desired solution viscosity. For example, a 2% W/V aqueous solution of Methocel™ E-15 has a viscosity of about 13-18 cps at 20° C.

A solid dosage form of the invention can be coated with a finish coat as is commonly done in the art to provide the desired shine, color, taste or other aesthetic characteristics. Materials suitable for preparing the finish coat are well known in the art and found in the disclosures of many of the references cited and incorporated by reference herein.

Various other components, in some cases not otherwise listed above, can be added to drug- or agent-containing formulations for optimization of a desired active agent release profile including, by way of example and without limitation, glycerylmonostearate, nylon, cellulose acetate butyrate, d,l-poly(lactic acid), 1,6-hexanediamine, diethylenetriamine, starches, derivatized starches, acetylated monoglycerides, gelatin coacervates, poly (styrene-maleic acid) copolymer, glycowax, castor wax, stearyl alcohol, glycerol palmitostearate, poly(ethylene), poly(vinyl acetate), poly(vinyl chloride), 1,3-butylene-glycoldimethacrylate, ethyleneglycol-dimethacrylate and methacrylate hydrogels.

It should be understood that compounds used in the formulation arts, including the art of pharmaceutical formulation, generally serve a variety of functions or purposes. Thus, whether a compound named herein is mentioned only once or is used to define more than one term herein, its purpose or function should not be construed as being limited solely to the named purpose(s) or function(s).

For preparing liquid or solid compositions such as tablets, the therapeutically active agent, including, for example, an opioid agonist, alone or in conjunction with an opioid antagonist, is mixed with a pharmaceutical carrier or excipient, such as conventional tableting ingredients and other pharmaceutical diluents, such as water, to form a solid intermediate composition containing a homogeneous mixture of a compound or a non-toxic pharmaceutically acceptable salt thereof. When referring to these intermediate compositions as homogeneous, it is meant that the therapeutically active agent(s), including, for example, an opioid antagonist, alone or in conjunction with an opioid agonist, is dispersed evenly throughout the composition so that the composition may be readily subdivided into equally effective unit dosage forms such as capsules, tablets, caplets, or pills. This solid preformulation composition is then subdivided into unit dosage forms of the type described above containing the above-stated dose of the therapeutically active agent(s), including, for example, an opioid antagonist, alone or in combination with opioid agonist.

For immediate release formulations, concurrent release and released concurrently refer to release in in vitro dissolution assays in an overlapping manner of more than one therapeutically active agent. The respective beginnings of release of each agent can but need not necessarily be simultaneous. Concurrent release will occur when the majority of the release of the first agent overlap a majority of release of the second agent. According to one exemplary embodiment, release of the agonist and antagonist begins and ends at approximately the same time. In some embodiments of formulations comprising an opioid antagonist and an opioid agonist, the dissolution rates of the antagonist and the agonist are substantially the same. A desired portion of each active pharmaceutical ingredient may be released within a desired time. The desired portions may be, for example, 5%, 50% or 90%, or some other percentage. The desired time may be, for example, 10 minutes, 20 minutes, 30 minutes or 45 minutes. Generally, the entire charge of each therapeutically active agent is released in less than 120 min, less than 90 min, less than 60 min, less than 45 min, less than 30 min, less than 20 min or less than 10 min. Preferably, the entire charge of each active pharmaceutical ingredient is released in less than 45 minutes.

Dosage forms of the present invention can be presented in any type of container-closure system or holding vessel of any type for packaging one or more gelatin capsules, including enrobed cores that are liquids, tablets or capsules. For example, a bottle, envelope, sachet, vial, tube, blister pack, bag, or pouch comprising essentially of the dosage forms presented herein are included. Various types of blister packs are described, for example, in U.S. Pat. No. 5,624,036, the disclosure of which is incorporated herein by reference. A non-limiting example of a blister pack includes push-through packs which are made with an aluminum foil or aluminum foil laminate lid. Blister packs can optionally contain materials of construction or design which may affect the stability, impart tamper evidency, or evidence of exposure to resist children's access or aid dispensation of the product from its primary container. For instance, they may protect dosage forms of the invention, including enrobed tablets and capsules from extraneous influences such as moisture, light, oxygen and dirt. The container-closure system may preserve a desired environment for the product within the package by its design or by inclusion of an additional component separate from the product such as a desiccant or humectant.

The container-closure system by its design or through incorporation of a component within may indicate or record exposure to certain conditions including temperature, humidity or vibration. Different container closure systems (bottles, blisters, pouches) are constructed of different materials and with various physical design that imparts function. For example, bottles may be glass (most protective) or plastic. There are several different plastics polymers that are commonly used including, for example polyethylene (low and high density), polypropylene, polyvinylidene fluoride (PVDF). Advantages of glass include its imperviousness to moisture and oxygen transmission. Bottles (glass and plastic) may incorporate colorants. Light blocking coatings, or pacifiers are useful in packaging to block transmission of light, moisture and/or oxygen. Bottles may include specialized seals, including foam, paper and foil seals within the closure that improve barrier to the above elements and are tamper evident. Foil seals (single or laminates, sealed by magnetic induction rather than adhesive) may be used for moisture barrier and tamper-evidence. Plastic bottles may also include additives to the polymer that opacifies the plastic (e.g., titanium dioxide) that, at certain levels, effectively minimize light transmission, and may also include a light blocking coating for the same reason. Bottles may incorporate a desiccant or humectant (packets or cartridges) within for humidity control.

Blisters may be constructed completely from foil, or from a plastic film closed with a foil lidding. Each of these materials may be included within a laminate structure, and may include polyester, polyethylene or polypropylene to prevent “push-through”, to impart child-resistance and a paper layer externally. All pharmaceutical packaging is child-resistant, but blisters may have additional design features that make them easy to open thus facilitating removal of medication by the elderly or physically challenged. Typical plastic films include those made from polyvinyl chloride (PVC), polyvinylidene chloride (PVdC) coated PVC, and polypropylene, vinyl/polyethylene/Aclar® laminate. Such films have different moisture and oxygen transmission characteristics. In addition to the type of plastic, film thickness influences transmissibility also. Certain of these films may be opacified as well. A desiccant can be contained within the blister package design, or within a pouch that contains the blister package. The foil blister may also incorporate a desiccant into its design for dehumidification. Accordingly, dosage forms of the invention are conveniently packaged for safety, stability and ease of use as described above.

### EXAMPLES

The following examples are provided for illustrative purposes and are not to be construed to limit the scope of the claims in any manner whatsoever.

### Example 1

### Controlled Release Core Dosage Formulations

The controlled release core of dosage forms according to the present invention can be in any type of pharmaceutically acceptable dosage form comprising at least one therapeutically active agent and at least one controlled release material. For example, the controlled release core can be in the forms of liquids, semi-solids and solids, including, pills, tablets, capsules and caplets. Preferred dosage forms for the controlled release core of the present invention are tablets and capsules. Preferred opioid agonists and, optionally, opioid antagonists of the controlled release core of the present invention include oxycodone, morphine, hydrocodone, tramadol, oxymorphone, hydromorphone, naltrexone, and nalmefene. For the purpose of illustration only, the following examples describe controlled release tablets and capsules comprising either oxycodone alone or in combination with naltrexone.

### Capsules: SAIB Liquids and SAIB Films

In an preferred aspect of the invention, an encapsulated liquid fill comprising oxycodone alone or optionally with naltrexone is mixed with SAIB, a high viscosity liquid controlled release material.

The dosage amounts of oxycodone alone or optionally, with naltrexone are described herein.

To achieve a particular desired viscosity in the resulting SAIB composition, the amount of SAIB and at least one biocompatible solvent, preferably ethanol, is optimized. For example, a low viscosity solution that can be expelled from a glass pipet is obtained with a mixture containing 9 g of SAIB combined with 1 g of ethanol whereas, a thin film that can retain its shape for more than one week is obtained with a mixture containing 8 g of SAIB combined with 1 g of ethanol.

In order to obtain a soluble liquid fill, the amounts of (i) SAIB, (ii) at least one biocompatible solvent, and (iii) at least one therapeutically active agent is optimized. For example, in formulations comprising small organic molecules such as ibuprofen, approximately 15% (by weight) of ethanol is added to achieve solubility with SAIB whereas, formulations comprising large peptidic molecules such as bovine serum albumin, do not solubilize with about 40% ethanol, even with the addition of co-solvents, such as glycerol and/or DMSO. Also, in some organic molecules formulations such as naproxen (sodium salt) another type of solvent, glycolfurol, is required to achieve solubility since naproxen is not soluble in ethanol and ethylacetate.

In a preferred aspect, the amounts of (i) SAIB, (ii) at least one biocompatible solvent and (iii) oxycodone alone or optionally, with naltrexone are optimized to achieve a desired viscosity. Preferred solvents for SAIB formulations with small organic molecules include, but are not limited to, ethanol, glycofurol, ethyllactate, ethylacetate, N-methyl-pyrrolidone, and propylene carbonate. Optionally, cosolvents, such as dimethylsulfoxide, or glycers may be added to enhance the solubility. However, the amount and type of solvent(s) with SAIB formulations is optimized with the oxycodone alone and optionally, naltrexone that is to be formulated. In this example, the amount of SAIB and the amount and type of biocompatible solvent(s) used with oxycodone alone or optionally, with naltrexone is optimized to produce a resultant liquid mixture of (i) SAIB, (ii) biocompatible solvent(s), and (iii) oxycodone alone or, optionally, with naltrexone, is pharmaceutically acceptable for encapsulation and/or tabulation.

In addition, at least one additive may be included in the oxycodone/SAIB or oxycodone/naltrexone/SAIB mixture to increase solubility. Such additives include, for example, cellulose acetate butyrate (CAB), cellulose acetate propionate (CAP), PVP, PVP-25, PEG-10K, PEG-1K, and sucrose. Again, the amount and type of additive(s) should be optimized with the particular type of therapeutic agent that is to be formulated. In a preferred aspect, the amount and type of additive(s) used with oxycodone alone or optionally, with naltrexone is optimized to produce a resultant liquid mixture of (i) SAIB, (ii) at least one biocompatible solvent, and (iii) oxycodone alone or, optionally, with naltrexone, and (iv) additive, wherein the mixture is pharmaceutically acceptable for encapsulation and/or tabulation

In another preferred aspect, the resultant liquid mixture of (i) SAIB, (ii) at least one biocompatible solvent, (iii) oxycodone alone or optionally, with naltrexone, and (iv) optionally, at least one additive is loaded into an aerosol container and sprayed onto agar plates to form an adhesive continuous film. In yet another embodiment, the resultant liquid mixture of (i) SAIB, (ii) at least one biocompatible solvent, (iii) oxycodone alone or optionally, with naltrexone, and (iv) optionally, at least one additive is sprayed onto gelatin. In yet a further embodiment, the resultant liquid mixture of (i) SAIB, (ii) at least one biocompatible solvent, (iii) oxycodone alone or optionally, with naltrexone, and (iv) optionally, at least one additive is loaded into a syringe equipped with a gauged needle and extruded.

### Capsules: Coated Beads

In an embodiment of the invention, oxycodone controlled release beads are incorporated into hard gelatin capsules which can then be encapsulated alone or optionally, with naltrexone controlled release beads. For instance, oxycodone controlled release beads are formulated and combined with naltrexone controlled release beads in a gelatin capsule.

In another embodiment of the invention, beads containing both oxycodone and naltrexone are incorporated into hard gelatin capsules which are then encapsulated. Thus, in this embodiment, encapsulation of one bead releases both oxycodone and naltrexone simultaneously.

The dosage amounts of oxycodone alone or optionally, with naltrexone are described in the specification.

In this non-limiting example, the controlled release beads are generated in a multi-step process wherein the controlled release materials are spray dried onto beads containing oxycodone and/or naltrexone. First, inert non-pareil beads (i.e. 30/35 mesh) are layered with oxycodone and/or naltrexone, by spray drying the beads with an aqueous solution of oxycodone and/or naltrexone in a fluid bed coater with Wurster insert. The non-pareil beads and/or the aqueous solution of oxycodone and/or naltrexone may contain excipients, such as Plasdone C30 and talc, binders, such as povidone and Eudragit RS30D, and fillers, such as lactose. Thus, the non-pareil beads can be spray dried, for instance, with a blend of (i) a binder solution of povidone and Eudragit RS30D and (ii) an aqueous solution of oxycodone and/or naltrexone.

Second, the oxycodone, naltrexone, or oxycodone/naltrexone beads are optionally sealed with an inert sealing solution, such as Opadry Clear (HPMC) solution.

Next, an aqueous sustained release solution is spray dried onto the sealed oxycodone, naltrexone, or oxycodone/naltrexone beads to produce the corresponding resultant controlled release beads. An example of an aqueous sustained release solution contains Eudragit R30SD, tributyl citrate, Tween 80, and talc. Another example of an aqueous sustained release solution contains Eudragit R30SD, Eudragit RL30D, triethyl citrate, talc, and triethyl citrate. Spray drying steps can be performed in a fluid bed coater with Wurster insert.

These beads can be optionally coated with additional Opadry Clear (HPMC) for further sealing and/or spray dried with an enteric coating composition. Both the Opadry Clear (HPMC) solution and an enteric coating composition are dissolved in aqueous solution before being used in the spray drying apparatus. Beads are then cured at elevated temperature for a period of time, so as to ensure complete drying of the beads.

Lastly, cured controlled release beads are encapsulated into suitably sized capsules. In an embodiment, oxycodone controlled release beads alone or, optionally, with naltrexone controlled release beads are encapsulated into hard gelatin capsules. In another embodiment, oxycodone/naltrexone controlled release beads are encapsulated into hard gelatin capsules.

Dissolution studies may be conducted on the resultant cured beads. Samples can be measured for the rate of dissolution using any spectroscopic measurement. For example, HPLC analysis of the dissolved beads monitoring the UV/vis characteristics of oxycodone or oxycodone/naltrexone can be measured over set increments of time to determine the rate of dissolution.

### Tablet: Dispersed Granulates

In an embodiment of the invention, controlled release granulates are combined with melted wax, such as cetostearyl alcohol, to produce waxed granulates that are subsequently milled and mixed with other excipients before finally being compressed into tablets. The controlled release granulates comprise an opioid agonist and optionally, opioid antagonist dispersed in a controlled-release matrix.

In a preferred aspect of the invention, the controlled release tablet comprises controlled release granulates which comprise oxycodone and optionally, naltrexone dispersed in a controlled-release matrix.

The dosage amounts of oxycodone alone or, optionally, with naltrexone are described in the specification.

In this non-limiting example, the controlled release granulates are generated in a multi-step process. First, the opioid agonist and optionally, opioid antagonist is dissolved in an aqueous solution before being granulated with a solution of spray dried lactose, hydroxyethyl cellulose, and optionally, either an opioid agonist or opioid agonist/antagonist.

Next, the resultant granulations are dried in a fluid bed dryer. The dried granulations are then passed through a mill and can be further dried before proceeding to the next step, waxing. The dried granulations can be waxed by adding melted cetostearyl alcohol to the granulations during the mixing step. Before passing onto a mill, the waxed granulates are cooled on a fluid bed dryer. The milled, waxed granulates can then be added with excipients, such as talc and magnesium stearate, before compression with a tablet press.

### Example 2

### Immediate Release Gelatin Capsule for Oral Dosage Form

Gelatin capsules comprising at least one therapeutically active agent are used to encase, enrobe, or encapsulate controlled release cores prepared, for example, according to Example 1. Hard or soft gelatin capsules can be used as the immediate release gelatin capsule. Soft gelatin capsules are preferred for the preparation of oral dosage forms according to the invention. Numerous methods for encapsulating the controlled release core are described, for example, in U.S. Pat. Nos. 5,146,730, 5,595,758, 6,482,516. A variety of methods and materials related to the preparation and use of gelatin formulations, coatings and capsules are described, for example, in U.S. Pat. Nos. 3,959,540; 4,744,988; 4,780,316; 5,200,191; 5,380,534; 5,422,160; 5,484,598; 5,505,961; 5,569,466; 5,595,758; 5,624,681; 5,682,733; 5,735,105; 5,750,145; 5,817,323; 5,827,535; 5,891,470; 5,985,321; 6,096,338; 6,120,806; 6,183,845; 6,193,999; 6,214,376; 6,251,426; 6,258,380; 6,285,380; 6,288,894; 6,387,400;

### Liquid Controlled Release Core

Where the controlled release core is in liquid form, the immediate release gelatin capsule can be a soft or hard gelatin capsule. Preferred soft gelatin capsules suitable for use in the immediate release gelatin capsule include Softlet® and Gelatin Binary System® from Banner Pharmacap and Liquid-Gels®, RP Scherersol®, and Puslin-Cap® from Cardinal/RP Scherer Corp. Methods for encapsulating a liquid fill formulant are well known and are described, for example, in U.S. Pat. No. 6,251,426.

Gelatin capsules compositions comprise gelatin of varying bloom strength and optionally further comprise at least one plasticizer, at least one gelatin extender, at least one additive, at least one colorant, at least one preservant, at least one surfactant, at least one drying agent, at least one taste modifier, at least one moisture retaining agent, and/or at least one opacifies. At least one therapeutically active agent is included in the composition of the formulation for the immediate release gelatin capsule. The at least one therapeutically active agent in the composition of the gelatin capsule formulation is (i) an opioid agonist alone, such as oxycodone, (ii) an opioid antagonist, such as naltrexone, or (iii) combination of an opioid agonist and opioid antagonist, such as oxycodone and naltrexone.

Gelatin capsule compositions comprising at least one therapeutically active agent is heated into a molten mass and is fed onto drums to form two spaced sheets or ribbons. The ribbons are fed around rollers and brought together at a convergent angle into the nip of a pair of roller dies. The liquid controlled release core is fed into the wedge-shaped joiner of the ribbons. The gelatin ribbons are continuously conveyed between the dies, with portion of the liquid controlled release core being trapped between the sheets inside the die cavities. The sheets are then pressed together to form a continuous gelatin covering around the entrapped liquid controlled release core to form resultant capsules. Capsules are open air or tumble-dried in a series of hollow drums with perforated walls that continuously pump heated dry air. Drying times span approximately 16-24 hours. After the capsules exit the last drying drum, the capsules are typically spread on drying trays are cooled. Cooled capsules are packaged in aluminum blistered foil packs.

### Tablet or Capsule Controlled Release Core

Where the controlled release core is a tablet or capsule, the immediate release gelatin capsule is enrobed over the tablet or capsule and can be in the form of a hard or soft gelatin capsule. Preferred soft gelatin capsules suitable for use in the immediate release gelatin capsule include Softlet® and Gelatin Binary System® from Banner Pharmacap and Liquid-Gels®, RP Scherersol®, and Puslin-Cap® from Cardinal/RP Scherer Corp. Methods for enrobing a tablet or capsule are well known and are described, for example, in U.S. Pat. No. 6,482,516. Enrobing methods produce tablets or capsules having soft elastic gelatin film sealed to opposite side of the tablet or core in an essentially edge-to-edge manner along a seal line.

Gelatin composition comprises gelatin of varying bloom strength and optionally further comprises at least one plasticizer, at least one gelatin extender, at least one additive, at least one colorant, at least one preservant, at least one surfactant, at least one drying agent, at least one taste modifier, at least one moisture retaining agent, and/or at least one opacifier. At least one therapeutically active agent is mixed in the gelatin capsule composition to be used in formulating the immediate release gelatin capsule. The at least one therapeutically active agent in the gelatin capsule composition is (i) an opioid agonist alone, such as oxycodone, (ii) an opioid antagonist, such as naltrexone, or (iii) combination of an opioid agonist and opioid antagonist, such as oxycodone and naltrexone.

Gelatin capsule compositions comprising at least one therapeutically active agent is heated into a liquid. Liquid gelatin capsule compositions are poured into a dispensing device and kept at an elevated temperature by an electric heater. The liquid gelatin is introduced to a moving casting surface as a layer of gelatin of predetermined thickness and solidifies on a drum casting surface sufficiently to form films. The gelatin film passes from individual tractor rolls and is wrapped around an adjacent die roll. core tablets or capsules are processed into a feed horn and placed symmetrically around the die roll. Heater blocks are placed as close as possible to the point at which the tablet or capsule core emerges from the wedge-shaped lower portion of the feed horn at the nip. The die nip is the place where films are brought into contact with each other so as to seal the film together around the tablet or capsule and cut the enrobed tablet or capsule from the film. Enrobed tablets or capsules are open air or tumble-dried in a series of hollow drums with perforated walls that continuously pump heated dry air. Drying times span approximately 16-24 hours. After the enrobed tablets or capsules exit the last drying drum, the capsules are typically spread on drying trays are cooled. Cooled enrobed tablets or capsules are packaged in aluminum blistered foil packs.

### Example 3

### Dosage Formulations with Commercially Available Controlled Release Therapeutically Active Agents

A variety of commercially available dosage form and controlled release formulations of therapeutically active agents, including opioid agonists, such as oxycodone, hydrocodone, and morphine, are useful as controlled release cores for the preparation of oral dosage forms according to the invention. Preferred commercial dosage forms and formulations of opioid agonists include, for example, OXYCONTIN® from Purdue Pharma, MS-CONTIN® from Purdue Frederick and AVINZA™ from Elan. Additional non-limiting examples of commercial controlled release formulations comprising opioid agonists include Ovamorph SR from Boehringer Ingelheim and Roxanol-SR and Kadian from Faulding. However, any commercial or non-commercial controlled release formulation of any therapeutically active agent, including any opioid agonist, can be used in the controlled release core according to the invention.

For example, OXYCONTIN® from Purdue Pharma is a controlled release tablet formulation comprising oxycodone hydrochloride in doses of 10 mg, 20 mg, 40 mg, 80 mg, and formerly 160 mg. OXYCONTIN® tablets are designed to provide controlled delivery of oxycodone over 12 hours in a pH independent manner. Oral bioavailability of oxycodone ranges from about 60% to about 87%. OXYCONTIN® tablets exhibit a biphasic absorption pattern with two apparent absorption half-times, t_{1/2}, of 0.6 and 6.9 hours, which described the initial release of oxycodone from the tablet followed by a prolonged release.

Additionally, for example, MS-CONTIN® from Purdue Frederick is a controlled release formulation comprising morphine sulfate in doses of 15, 30, 60, 100, and 200 mg. MS-CONTIN® tablets are designed to provide controlled delivery of morphine over 12 hours. Average tₘₐₓ for MS-CONTIN® tablets is approximately 2.06 hours and average half-life of absorption, t_{1/2}, is 0.87 hours.

Additionally, for example, AVINZA™ from Elan is an extended release capsule formulation comprising morphine sulfate in doses of 30, 60, 90, and 120 mg. AVINZA™ capsules contain both immediate release and extended release beads of morphine to achieve plateau morphine plasma concentrations throughout a 24-hour dosing interval. Following a single-dose of 60 mg of AVINZA™ under fasting conditions, morphine concentration of approximately 3 to 6 ng/mL were achieved within 30 minutes and maintained for 24 hours.

Commercial formulations of controlled release opioid agonists, preferably OXYCONTIN®, MS-CONTIN®, and AVINZA™ can be enrobed, encased, or encapsulated according to Example 2 with an immediate release gelatin capsule comprising at least one therapeutically active agent, preferably an opioid agonist, an opioid antagonist, or a combination of an opioid agonist and antagonist present in preferred amounts disclosed herein.

Where the controlled release core is a commercial or non-commercial controlled release dosage form or formulation comprising an opioid agonist, the therapeutically active agent of the immediate release gelatin capsule can be at least one opioid agonist present in amounts within preferred ranges disclosed herein, including, for example, from about 0.025 mg to about 60 mg. Where the controlled release core is OXYCONTIN®, the immediate release gelatin capsule comprises oxycodone. Where the core is 10 mg OXYCONTIN® tablet, the amount of oxycodone in the gelatin capsule enrobing the tablet is from about 0.25 mg to about 2.0 mg. Where the controlled release core is MS-CONTIN® or AVINZA™ (as a hydrochloride or free base), the immediate release gelatin capsule coating comprises morphine sulfate. Where the core is a 30 mg MS-CONTIN® tablet or a 30 mg AVINZA™ capsule, the amount of morphine sulfate in the gelatin capsule enrobing the tablet or capsule is from about 0.75 mg to about 60 mg.

Additionally, where the controlled release core is a commercial or non-commercial controlled release formulation comprising an opioid agonist, the therapeutically active agent of the immediate release gelatin capsule can be at least one opioid antagonist present in amounts within preferred ranges disclosed herein, including, for example, from about 0.000001 to about 0.5 mg. Where the controlled release core is OXYCONTIN®, the immediate release gelatin capsule coating comprises naltrexone or nalmefene. Where the controlled release core is MS-CONTIN®or AVINZA™, the immediate release gelatin capsule coating comprises naltrexone or nalmefene.

Additionally, where the controlled release core is a commercial or non-commercial controlled release formulation comprising an opioid agonist, the therapeutically active agent of the immediate release gelatin capsule can be at least one opioid agonist and at least one opioid antagonist present in amounts within preferred ranges disclosed herein. Where the controlled release core is OXYCONTIN®, the immediate release gelatin capsule coating comprises oxycodone in an amount ranging from about 0.025 to about 60 mg and naltrexone or nalmefene in an amount ranging from about 0.000001 to about 0.5 mg. Where the controlled release core is MS-CONTIN® or AVINZA™, the immediate release gelatin capsule coating comprises oxycodone in an amount ranging from about 0.025 to about 60 mg and naltrexone or nalmefene in an amount ranging from about 0.000001 to about 0.5 mg.

### Example 4

### In Vivo Testing of Oral Dosage Formulations in Dog Models

In vivo testing in dog models is performed to determine the relative bioavailability of various oral dosage formulations presented herein. Studies using common mammalian laboratory animals, such as dogs, are essential and are routinely used for the evaluation of absorption, distribution, metabolism, and excretion (ADME) properties of chemical entities. The dog is selected for this study based on anatomical, physiological, and biochemical similarities to human, which may facilitate extrapolation of observed ADME properties to man.

This study uses the minimum number of animals required for complete collection of the desired biological samples and to obtain scientifically valid results. This study is conducted in accordance with applicable Standard Operating Procedures and generally recognized good laboratory practice. All procedures in the protocol of this study are in compliance with the Animal Welfare Act Regulations as set forth in 9 C.F.R. 3. All personnel involved in this study will follow all safety precautions as required by Testing Laboratory's Policies and Procedures in consideration of the Material Safety Data Sheet or other relevant safety information. Animals are maintained and monitored for good health in accordance with laboratory Standard Operating Procedures and at the discretion of a laboratory animal veterinarian.

Six healthy female purebred beagles from a stock colony weighing approximately 5 kg to 7 kg and approximately 4 months to 18 months in age are entered into a 4-phase study. Each phase is followed by a 14-day washout period thus Phase 1 is administered on Day 1, phase 2 is administered on Day 14, phase 3 is administered on Day 28, and phase 4 is administered on Day 42. As outlined in Table 1, formulations A, B, C, and D will be administered as oral capsule doses to each dog, followed by administration of a placebo capsule. Animals are fasted overnight prior to dosing through approximately 4 hours post-dose for each phase of the study. Individual doses for each dog are calculated based on body weight taken on each day of dosing. Prior to and after oral dose administration of various oral dosage forms according to the invention (post dose samples withdrawn at 0.167, 0.33, 0.67, 1, 1.5, 2, 3, 4, 6, 8, 12, 24, 48, 72, and 96 hours after administration), approximately 1 mL of blood from each dog is collected into tubes containing heparin anticoagulant. Blood samples are stored on wet ice, in chilled Kryorack, or at approximately 5° C. prior to centrifugation to obtain plasma. Resultant plasma samples are tested for the presence of the administered therapeutically active agent(s) using analytical procedures known in the art.

Each dog is uniquely marked with a numbered ear tattoo for proper identification. The dogs are acclimated in an environment-controlled study room (temperature of 18° C.-29° C., 12-hour light/12-hour dark cycle) for at least 4 days prior to the initial dose administration. During acclimation and the test period, the dogs are housed in individual cages and are not commingled in order to minimize the possibility of injury. The dogs are fed non-certified canine diet #5L03 (PMI Feeds, Inc.) ad libitum, except as specified under Dosing Procedures, and may be provided with certified canine treats, as appropriate, during non-fasted periods. The dogs are provided ad libitum with tap water from a well supply that is tested quarterly and annually for total coliforms and for the presence of pesticides, trace metals, and heavy metals to ensure safe drinking status. Both the food and water given to the dogs do not contain any known contaminants that would interfere with the conducted study.

Mortality and moribundity checks are performed twice daily, in the morning and evening. Cageside observation for general health and appearance is done once daily. Any unusual observations noted during dose administration and sample collection are recorded in the raw data. Body weights are taken on each day of dose administration.

The invention now being fully described, it will be apparent to one of ordinary skill in the art that many changes and modifications can be made thereto without departing from the spirit or scope of the appended claims.

1: An oral dosage form comprising (i) an controlled release core; and (ii) an immediate release gelatin capsule around the controlled release core, wherein the controlled released core comprises at least one therapeutically active agent and at least one controlled release material; and wherein the immediate release gelatin capsule comprises at least one therapeutically active agent.

2-92. (canceled)

93. A method of making an oral dosage form comprising:
(i) preparing a controlled release core, wherein the controlled released core comprises at least one therapeutically active agent and at least one controlled release material; and
(ii) an immediate release gelatin capsule around the controlled release core, wherein the immediate release gelatin capsule coating comprises at least one therapeutically active agent.

94. (canceled)

95. A method of selectively enhancing analgesic potency of an opioid agonist or attenuating an adverse side effect of the opioid agonist in a human subject comprising:
(i) administering to the human subject an oral dosage form comprising:
(a) a controlled release core; and
(b) an immediate release gelatin capsule around the controlled release core;
wherein the controlled released core comprises at least one opioid agonist and at least one controlled release material;
wherein the immediate release gelatin capsule coating comprises at least one opioid antagonist, and wherein the antagonist enhances the analgesic potency of the opioid agonist or attenuates an adverse side effect of the agonist in the human subject.

96. (canceled)

97. A method of selectively enhancing analgesic potency of an opioid agonist or attenuating an adverse side effect of the opioid agonist in a human subject comprising:
(i) administering to the human subject an oral dosage form comprising:
(a) a controlled release core; and
(b) an immediate release gelatin capsule around the controlled release core;
wherein the controlled released core comprises at least one opioid agonist and at least one controlled release material;
wherein the immediate release gelatin capsule comprises at least one opioid agonist and at least one opioid antagonist, and wherein the antagonist enhances the analgesic potency of the opioid agonist or attenuates an adverse side effect of the agonist in the human subject.

98. (canceled)

99. A method of selectively enhancing analgesic potency of an opioid agonist or attenuating an adverse side effect of the opioid agonist in a human subject comprising:
(i) administering to the human subject an oral dosage form comprising:
(a) a controlled release core; and
(b) an immediate release gelatin capsule coating around the controlled release core;
wherein the controlled released core comprises at least one opioid 30 agonist, at least one opioid antagonist, and at least one controlled release material;
wherein the immediate release gelatin capsule comprises at least one opioid agonist, and wherein the antagonist enhances the analgesic potency of the opioid agonist or attenuates an adverse side effect of the agonist in the human subject.

100. (canceled)

101. A method of selectively enhancing analgesic potency of an opioid agonist or attenuating an adverse side effect of the opioid agonist in a human subject comprising:
(i) administering to the human subject an oral dosage form comprising:
(a) a controlled release core; and
(b) an immediate release gelatin capsule around the controlled release core;
wherein the controlled released core comprises at least one opioid agonist, at least one opioid antagonist, and at least one controlled release material;
wherein the immediate release gelatin capsule comprises at least one opioid agonist and at least one opioid antagonist, and wherein the antagonist enhances the analgesic potency of the opioid agonist or attenuates an adverse side effect of the agonist in the human subject.

102-104. (canceled)
